(19) United States
(12) Patent Application Publication
(10) Pub. No.: US 2011/0008465 A1
(43) Pub. Date: Jan. 13, 2011

(54) SESQUITERPENE FORMULATIONS, KITS AND METHODS OF USE THEREOF

(76) Inventors: Jean Legault, Chicoutimi (CA); Andre Pichette, Chicoutimi (CA); Serge Lavoie, Chicoutimi (CA)

Correspondence Address: GOUDREAU GAGE DUBUC, 2000 MCGILL COLLEGE, SUITE 2200, MONTREAL, QC, H3A 3H3, CA

(21) Appl. No.: 12/602,382

(22) PCT Filed: Jun. 2, 2008
(86) PCT No.: PCT/CA08/01063

§ 371 Date: Sep. 28, 2010

Related U.S. Application Data
(60) Provisional application No. 60/941,117, filed on May 31, 2007.

(30) Foreign Application Priority Data
May 6, 2008 (CA) CA2008000865

(51) Int. Cl. A61K 31/015; A61K 31/337; A61K 31/475; A61K 31/282; A61K 31/175; A61K 31/196; A61K 33/24; A61K 31/4164; A61K 31/198; A61K 31/704; A61K 31/136; A61K 31/7048; A61K 31/513; A61K 31/138; A61K 31/525; A61P 35/00

(52) U.S. Cl. 424/649; 514/766; 514/449; 514/283; 514/492; 514/590; 514/567; 514/398; 514/564; 514/34; 514/662; 514/27; 514/274; 514/648; 514/249

(57) ABSTRACT

A pharmaceutical composition comprising a water insoluble sesquiterpene, one or more antioxidants and one or more solubilizers selected from the group consisting of an oil, PEG400, a derivative of castor oil and ethylene oxide, and polysorbate 80, and methods of use thereof.

### CROSS REFERENCE TO RELATED APPLICATIONS

This application claims priority under 35 U.S.C. §119(e), of U.S. provisional application Ser. No. 60/941,117, filed on May 31, 2007. This document is incorporated herein in its entirety by reference.

### FIELD OF THE INVENTION

The present invention relates to sesquiterpenes formulations, kits and methods of use thereof.

### BACKGROUND OF THE INVENTION

Sesquiterpenes are present in essential oils of certain plants including balsam fir, clove bud and hop.

It was recently discovered that certain sesquiterpenes have interesting biological activities. For instance, the in vitro cytotoxicity of certain anti-tumoral agents such as paclitaxel, docetaxel, cisplatine, and vinorelbine was shown to be improved with beta-caryophyllene.

Formulating such molecules into a pharmaceutical formulation presents important challenges. In particular, certain sesquiterpenes known for their therapeutic value have a very weak hydrosolubility, their molecular structure being devoid of hydrophilic moiety.

A frequently used method of formulating a weakly hydrosoluble molecule in an aqueous carrier involves the use of ethanol, an organic solvent that is appropriate for intravenous injectable formulation when it is used at small concentrations. This approach however disadvantageously results in a rapid phase separation when water is added to comply with the FDA requirement that ethanol be contained at a maximum concentration of 80%.

There is a need for an improved formulation for weakly hydrosoluble sesquiterpenes.

The present description refers to a number of documents, the content of which is herein incorporated by reference in their entirety.

### SUMMARY OF THE INVENTION

The present invention provides a stable formulation for water insoluble sesquiterpenes such as beta-caryophyllene.

The Applicants have discovered that beta-caryophyllene is sensitive to acidity.

They have also surprisingly discovered that in certain solubilizers found to be appropriate for liquid formulations, beta-caryophyllene ((1R,4E,9S)-4-11,11-trimethyl-8-methylenebicyclo[7.2.0]undec-4-ene, CAS registry number [87-44-5], FIG. 1) oxidizes into beta-caryophyllene oxide. Beta-caryophyllene oxide is considered to be an irritant and has no observed potentializing activity and is thus considered herein to be an impurity. In accordance with the present invention, the concentration of such an impurity in injectable solutions is low.

More specifically, in accordance with the present invention, there is provided a pharmaceutical composition comprising a water insoluble sesquiterpene, one or more antioxidants and one or more solubilizers selected from the group consisting of PEG400, an animal or vegetable oil (e.g., olive oil), a derivative of castor oil and ethylene oxide, and polysorbate 80.

In specific embodiments of the pharmaceutical composition, the sesquiterpene is beta-caryophyllene. In other specific embodiments of the pharmaceutical composition, the sesquiterpene is humulene. In other specific embodiments of the pharmaceutical composition, the sesquiterpene is farnesol. In other specific embodiments of the pharmaceutical composition, the sesquiterpene is nerolidol. In other specific embodiments of the pharmaceutical composition, the sesquiterpene is farnesylic acid. In other specific embodiments of the pharmaceutical composition, the sesquiterpene is torilin. In other specific embodiments of the pharmaceutical composition, the sesquiterpene is isocaryophyllene. In other specific embodiments of the pharmaceutical composition, the sesquiterpene is bisabolol.

In other specific embodiments of the pharmaceutical composition, the one or more antioxidants are selected from the group consisting of vitamin E, a hydrophilic vitamin E analog, alpha tocopherol acetate, butylated hydroxytoluene (BHT) and butylated hydroxyanisole (BHA). In other specific embodiments of the pharmaceutical composition, the antioxidant is 6-Hydroxy-2,5,7,8-tetramethylchroman-2-carboxylic acid. In other specific embodiments of the pharmaceutical composition, the antioxidant is vitamin E. In other specific embodiments of the pharmaceutical composition, the solubilizer is an animal or vegetable oil. In other specific embodiments of the pharmaceutical composition, the oil is olive oil. In other specific embodiments of the pharmaceutical composition, the solubilizer is a polysorbate. In other specific embodiments of the pharmaceutical composition, the polysorbate is polysorbate 80. In other specific embodiments of the pharmaceutical composition, the solubilizer is a derivative of castor oil and ethylene oxide.

In other specific embodiments, the pharmaceutical composition further comprises one or more isotonic agents selected from the group consisting of dibasic sodium phosphate, sodium bicarbonate, calcium chloride, potassium chloride, sodium lactate, glycerol, sorbitol, xylitol, sodium chloride, dextrose, a Ringer's solution, a lactated Ringer's solution and a mixture of dextrose and a mixture thereof. In other specific embodiments, the pharmaceutical composition comprises from about 0.01 mg/mL to about 100 mg/mL of beta-caryophyllene, from about 0.0001% to about 5% v/v of antioxidant, from about 0.01% to about 20% v/v of solubilizer, and an isotonic agent. In other specific embodiments, the pharmaceutical composition comprises about 1% v/v of beta-caryophyllene, about 0.1% v/v of antioxidant, about 5% v/v of solubilizer, and about 93.5% v/v of an isotonic agent. In other specific embodiments of the pharmaceutical composition, the antioxidant is vitamin E and the solubilizer is polysorbate 80. In other specific embodiments of the pharmaceutical composition, the antioxidant is 6-Hydroxy-2,5,7,8-tetramethylchroman-2-carboxylic acid and the solubilizer is polysorbate 80. In other specific embodiments of the pharmaceutical composition, the antioxidant is a combination of 6-Hydroxy-2,5,7,8-tetramethylchroman-2-carboxylic acid and of vitamin E. In other specific embodiments of the pharmaceutical composition, the isotonic agent is sodium chloride. In other specific embodiments of the pharmaceutical composition, the antioxidant is 6-Hydroxy-2,5,7,8-tetramethylchroman-2-carboxylic acid, the solubilizer is polysorbate 80 and the isotonic agent is sodium chloride. In other specific embodiments of the pharmaceutical composition, the antioxidant is vitamin E, the solubilizer is polysorbate 80 and the isotonic agent is sodium chloride.

In other specific embodiments of the pharmaceutical composition, the composition is an oral formulation. In other specific embodiments of the pharmaceutical composition, the oral formulation is a capsule. In other specific embodiments of the pharmaceutical composition, the composition is in a soft gel capsule. In other specific embodiments of the pharmaceutical composition, the composition has an enteric coating.

In other specific embodiments of the pharmaceutical composition, the oral formulation is an oil-based syrup. In other specific embodiments of the pharmaceutical composition, the syrup comprises olive oil as a solubilizer and vitamin E as an antioxidant.

In other specific embodiments, the pharmaceutical composition is in a daily dosage comprising from about 0.001 mg/kg to about 300 mg/kg of sesquiterpene. In other specific embodiments, the pharmaceutical composition is in a daily dosage comprising from about 0.001 mg/kg to about 40 mg/kg of sesquiterpene. In other specific embodiments, the pharmaceutical composition is in a daily dosage comprising from about 0.01 mg/kg to about 20 mg/kg of beta-caryophyllene. In other specific embodiments, the pharmaceutical composition is in a daily dosage comprising from about 0.5 mg/kg to about 4 mg/kg of beta-caryophyllene. In other specific embodiments, the pharmaceutical composition is in a daily dosage comprising about 0.5 mg/kg to about 2 mg/kg of beta-caryophyllene. In other specific embodiments, the pharmaceutical composition is in a daily dosage comprising about 1 mg/kg to about 4 mg/kg of beta-caryophyllene. In other specific embodiments, the pharmaceutical composition is in a daily dosage comprising from about 0.01 mg/kg to about 20 mg/kg of beta-caryophyllene. In other specific embodiments, the pharmaceutical composition is in a daily dosage comprising from about 0.5 mg/kg to about 4 mg/kg of beta-caryophyllene. In other specific embodiments, the pharmaceutical composition is in a daily dosage comprising about 0.5 mg/kg to about 2 mg/kg of beta-caryophyllene.

In other specific embodiments, the pharmaceutical composition further comprises an antitumoral agent. In more specific embodiments, the antitumoral agent is selected from the group consisting of alkylating agent, antimetabolite, antimitotic, antibiotic, topoisomeras II inhibitor, kinase inhibitors, a vinca alkaloid, immunotherapy and hormone. In other more specific embodiments, the antitumoral agent is an alkylating agent selected from the group consisting of carboplatin, melphalan, cyclophosphamide, lomustine, chlorambucil, carmustine and cisplatine. In other more specific embodiments, the antitumoral agent is a topoisomerase II inhibitor selected from the group consisting of etoposide, mitoxantrone, daunorubicin and doxorubicin. In other more specific embodiments, the antitumoral agent is an antimetabolite selected from the group consisting of 5-5-fluorouracil, floxuridine, gemcitabine, mercaptopurine, tioguanine, fludarabine, cytarabine, pemetrexed, raltitrexed and methotrexate. In other more specific embodiments, the antitumoral agent is an antimitotic selected from the group consisting of paclitaxel and docetaxel. In other more specific embodiments, the antitumoral agent is a vinca alkaloid selected from the group consisting of vinblastine, vincristine and vindesine, vinorelbine. In other more specific embodiments, the antitumoral agent is an antibiotic selected from the group consisting of aclarubicin and mitomycin C. In other more specific embodiments, the antitumoral agent is a kinase inhibitor selected from the group consisting of tamoxiphen and tyrphostin. In other more specific embodiments, the antitumoral agent is a hormone selected from the group consisting of steroid and glucocordicoid hormone. In other more specific embodiments, the antitumoral agent is paclitaxel. In other more specific embodiments, the antitumoral agent is docetaxel.

In accordance with another aspect of the present invention, there is provided a method of using the pharmaceutical composition of the present invention comprising administering the composition to a subject in need thereof.

In accordance with another aspect of the present invention, there is provided a method of using the pharmaceutical composition of the present invention comprising administering the composition to a subject in need thereof prior to administration of an antitumoral agent.

In accordance with another aspect of the present invention, there is provided a method of using the pharmaceutical composition of the present invention comprising administering the composition to a subject in need thereof after administration of an antitumoral agent.

In accordance with another aspect of the present invention, there is provided a method of using the pharmaceutical composition of the present invention comprising administering the composition to a subject in need thereof simultaneously to administration of an antitumoral agent.

In a specific embodiment of the method of the present invention, the antitumoral agent is selected from the group consisting of alkylating agent, antimetabolite, antimitotic, antibiotic, topoisomeras II inhibitor, kinase inhibitors, vinca alkaloid, immunotherapy and hormone. In another specific embodiment of the method of the present invention, the antitumoral agent is an alkylating agent selected from the group consisting of carboplatin, melphalan, cyclophosphamide, lomustine, chlorambucil, carmustine and cisplatine. In another specific embodiment of the method of the present invention, the antitumoral agent is a topoisomerase II inhibitor selected from the group consisting of etoposide, mitoxantrone, daunorubicin and doxorubicin. In another specific embodiment of the method of the present invention, the antitumoral agent is an antimetabolite selected from the group consisting of 5-5-fluorouracil, cytarabine and methotrexate. In another specific embodiment of the method of the present invention, the antitumoral agent is an antimitotic selected from the group consisting of paclitaxel and docetaxel. In another specific embodiment of the method of the present invention, the antitumoral agent is a vinca alkaloid selected from the group consisting of vinblastine, vincristine, vindesine and vinorelbine. In another specific embodiment of the method of the present invention, the antitumoral agent is an antibiotic selected from the group consisting of aclarubicin and mitomycin C. In another specific embodiment of the method of the present invention, the antitumoral agent is a kinase inhibitor selected from the group consisting of tamoxiphen and tyrphostin. In another specific embodiment of the method of the present invention, the antitumoral agent is a hormone selected from the group consisting of steroid and glucocordicoid hormone. In another specific embodiment of the method of the present invention, the antitumoral agent is paclitaxel. In another specific embodiment of the method of the present invention, the antitumoral agent is docetaxel.

In another specific embodiment of the method of the present invention, the step of administering the composition is performed intravenously. In another specific embodiment of the method of the present invention, the step of administering the composition is performed orally.

In another specific embodiment of the method of the present invention, the subject has a cancer selected from the group consisting of prostate cancer, breast cancer, small cell lung carcinoma, non-small cell lung carcinoma, colon adenocarcinoma, rectum cancer, non-Hodgkin's lymphoma, bladder cancer, kidney cancer, leukemia, mouth cancer, oesophagus cancer, larynx cancer, stomach cancer, melanoma, pancreatic cancer, endometrial cancer, uterine sarcoma, ovarian cancer, testicular cancer, multiple myeloma, brain tumor, thyroid cancer, Hodgkin's lymphoma, liver cancer, osteosarcoma and glioma. In another specific embodiment of the method of the present invention, the subject has a cancer selected from the group consisting of lung carcinoma and melanoma.

In accordance with another aspect of the present invention, there is provided a kit comprising the pharmaceutical composition of the present invention and instructions to use it in combination with an antitumoral agent.

In accordance with another aspect of the present invention, there is provided a use of the pharmaceutical composition of the present invention in the manufacture of a medicament. In a specific embodiment, the use is for the manufacture of a medicament for potentiating an antitumoral agent. In another specific embodiment, the use is for the manufacture of a medicament for treating cancer. In another specific embodiment, the pharmaceutical composition is used as a potentiator for an antitumoral agent. In another specific embodiment, the pharmaceutical composition is used as an antitumoral agent.

In accordance with another aspect of the present invention, there is provided a process for making a pharmaceutical composition of the present invention, comprising (a) mixing one or more antioxidants and one or more solubilizers to form a homogenous mixture; and (b) adding one or more water insoluble sesquiterpenes.

The articles “a,” “an” and “the” are used herein to refer to one or to more than one (i.e., to at least one) of the grammatical objects of the article.

The terms “including” and “comprising” are used herein to mean, and reused interchangeably with, the phrases “including but not limited to” and “comprising but not limited to”.

The term “such as” is used herein to mean, and is used interchangeably herein with, “such as but not limited to”.

The term “subject” in the context of the present invention relates to any mammal including a mouse, rat, pig, monkey and horse. In a specific embodiment, it refers to a human.

The terms “water insoluble sesquiterpene” are used herein to refer to a therapeutically useful water insoluble sesquiterpenes. A number of such therapeutically useful water insoluble sesquiterpenes are known in the art. They include beta-caryophyllene (FIG. 1), alpha-caryophyllene/humulene, isocaryophyllene, farnesol, nerolidol, farnesylic acid, (3E,5E)-3,7,11-trimethyl-9-oxododeca-1,3,5-triene, (2E,4E)-2,6,10-trimethylundeca-2,4,9-trienal, alpha-bisabolol, curcuphenol, curcudiol, vernolide, metachromin (sesquiterpene hydroquinone), hippochromin (sesquiterpene hydroquinone), zerumbone, torilin, costunolide, 8-epi-xanthatin, 8-epi-xanthatin epoxide, parthenolide, michelenolide, epoxygermacronolide, tithofolinolide, germacronolide, thapsigargin, xenitorin, parviflorene, suberosol, buddledin, suberosenone, helenalin, leitneridanin, illudin, hydroxymethylacylfulvene, 6-hydroxymethylacylfulvene, isodrimeninol, quadrone, terrecyclic acid A, isoquadrone, suberosenone, trichodermol, loukacinol and artemisinin. They also include their metabolites such as hydroxycaryophyllenes, caryophyllene oxides and hydroxycaryophyllene oxides. In specific embodiment, the formulation comprises one or more sesquiterpenes. In other embodiments, the formulation contains a single terpene. In more specific embodiment, the formulation comprises a single sesquiterpene.

The pharmaceutical compositions of the present invention preferably comprise one or more purified sesquiterpenes. As used herein, the term “purified” refers to a molecule (i.e., a sesquiterpene such as beta-caryophyllene) having been separated from one or more components of the composition in which it was originally contained (e.g., natural extracts or chemical synthesis contaminants). Hence, a “purified sesquiterpene” molecule is a molecule that is lacking in most other components (e.g., 70, 75, 80, 85, 90, 95, 96, 97, 98, 99, 100% free of contaminants). “Substantially pure sesquiterpene” is intended to include β-caryophyllene molecules that are at least 95% free of contaminants. The terms “purified sesquiterpene” or “substantially pure sesquiterpene” are intended to include both sesquiterpene purified from natural extracts and chemically synthesized sesquiterpene. By opposition, the term “crude” or “semi-purified” means molecules that have not been separated from other components of the composition from which the sesquiterpene originates (e.g., semi purified natural extracts, essential oils etc.). For the sake of brevity, the units (e.g. 66, 67 . . . 81, 82, 91, 92% . . . ) have not been specifically recited but are considered nevertheless within the scope of the present invention. Of course, a person skilled in the art would appreciate that in the context of pharmaceutical compositions it is preferable, although not essential, that the sesquiterpene be as pure as possible (i.e., substantially free of contaminants). Purity can be measured using any appropriate method such as by column chromatography, HPLC, etc.

### Dosage

Any amount of a pharmaceutical composition can be administered to a subject. The dosages will depend on many factors including the mode of administration and the age of the subject. Typically, the amount of one or more sesquiterpenes contained within a single dose will be an amount that effectively prevents, delays or reduces tumor in combination with an antitumoral agent administered before, in combination with or after sesquiterpene without inducing significant toxicity. As used herein the term “therapeutically effective amount” is meant to refer to an amount effective to achieve the desired therapeutic effect while avoiding adverse side effects. Typically, in accordance with the present invention, a sesquiterpene can be administered to subjects in doses ranging from 0.001 to 300 mg of sesquiterpene per kg of body weight each day and, in a more specific embodiment, 0.05 mg/kg/day to about 40 mg/kg/day. The dosage will be adapted by the clinician in accordance with conventional factors such as the extent of the disease and different parameters from the patient and will depend on the amount of antitumoral agent. Without being so limited, it is assumed that a perfusion can administer about 200 ml/hour for up to about 5 hours to an average adult of about 60 kg. Without being so limited, it is assumed that injections can administer as much as 1000 ml within 20 minutes. In accordance with a specific embodiment, formulations of the present invention contain about 10 mg/ml of beta-caryophyllene. 1000 ml of such formulation injected to an average adult weighing 60 kg contain 10 000 mg of beta-caryophyllene, namely 167 mg/kg.

The therapeutically effective amount of the pharmaceutical composition of the present invention may also be measured directly. The effective amount may be given daily or weekly or fractions thereof. Typically, a pharmaceutical composition of the invention can be administered in an amount providing about 0.001 up to about 300 mg of sesquiterpene per kg of body weight each day (e.g., 0.001, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 10, 15, 20, 25, 30, 50, 100, 200 or 300 mg/kg/day). Dosages may be provided in either a single or multiple dosage regimens. For example, in some embodiments the effective amount is a dose that ranges from about 0.01 to about 10 mg/kg/day, about 0.01 to about 5 mg/kg/day, from about 0.02 to about 1 mg/kg/, about 0.02 to about 2 mg/kg/day, about 0.02 to about 3 mg/kg/day, about 0.02 to about 4 mg/kg/day, about 0.14 to about 35 mg/kg/week, about 0.14 to about 42 mg/kg/week, about 0.14 to about 49 mg of the sesquiterpene every other day. In specific embodiments, beta-caryophyllene used to potentiate Taxotere™ is administered in a dosage of about 0.5 to about 2 mg/kg to a human. An average human adult would thus receive about 30 to about 120 mg and thus about 3 to 12 mL of a beta-caryophyllene formulation at a concentration of 10 mg/mL. Similarly, in other specific embodiments, beta-caryophyllene used to potentiate paclitaxel is administered in a dosage of about 1 to about 4 mg/kg to a human. An average human adult would thus receive about 60 to about 240 mg and thus about 6 to 24 mL of a beta-caryophyllene formulation at a concentration of 10 mg/mL.

These are simply guidelines since the actual dose must be carefully selected and titrated by the attending physician based upon clinical factors unique to each patient. The optimal daily dose will be determined by methods known in the art and will be influenced by factors such as the age of the patient as indicated above and other clinically relevant factors. In addition, patients may be taking medication for other diseases or conditions.

### Carriers/Vehicles

Pharmaceutical compositions of the present invention can be administered by routes such as orally, nasally, intravenously, intramuscularly, subcutaneously, intrathecally, intraperitoneally, intratumorally or intradermally. The route of administration can depend on a variety of factors, such as the environment and therapeutic goals.

Solubilizing agents useful for the present invention encompass one or more of polyoxyethylene-sorbitan-fatty acid esters, polyoxyethylene fatty acid esters, PEG glyceryl fatty acid esters, propylene glycol mono- or di-fatty acid esters, sorbitan fatty acid esters, polyoxyethylene-polyoxypropylene co-polymers, glycerol triacetate, monoglycerides, acetylated monoglycerides, polysorbate glycerol fatty acid esters, acetylated esters of fatty acids, acacia, carbomer copolymer, carbomer interpolymer, cholesterol, diethanolamine aluminium monostearate, carboxy methyl cellulose, sodium desoxycholate, egg yolk phospholipid, hydrolyzed gelatin, lecithin, lanolin alcohols, poloxamer, povidone, sodium dodecyl sulphate, sorbitol, oils such as vegetable oils or animal oils (see relevant sections of USP-NF and Nema, 1997). Non-limiting examples of vegetable oils include canola, corn, flax seeds, cotton seeds, soybean, walnut, pine nut, peanut, grape seed, sunflower, safflower, olive, coconut, palm oil etc). Non-limiting examples of animal oils include fish, seal oil and castor oil. Of course a combination of one or more solubilizing agents may be used in accordance with the present invention.

In more specific embodiments, the pharmaceutical composition includes any polysorbate including polysorbates 20, 21, 40, 60, 61, 65, 80, 81 and 85; Brij™ (polyoxyethyleneglycol alkyl ether having a polar side of 10 to 100 monomers) and Cremophor™ (e.g., Cremophor™ EL (derivative of castor oil and ethylene oxide); Cremophor™ A6 (Polyethylene glycol 260 mono(hexadecyl/octadecyl)ether and 1-Octadecanol) and Cremophor™ A25 (Polyethylene glycol 1100 mono(hexadecyl/octadecyl)ether).

The solubilizers containing polyoxyethylene chains such as polysorbates, PEG, and Brij™ are susceptible to formation of peroxides by radicalar reactions catalyzed by light and oxygen. In specific embodiments, solubilizers used in beta-caryophyllene formulations are PEG400, Cremophor™ EL, polysorbate 60 and polysorbate 80.

Antioxidants useful for formulations of the present invention include plant extracts (i.e. fruit, vegetable and/or leguminous extracts), algae extracts, microorganisms extracts such as yeast extracts and its derivatives, ferments, proteolysis hydrolysates, peptides, animal derivative extracts and synthetic compounds. More particularly, such ingredients include Ethylbisiminomethylguaiacol manganese chloride; dipalmitoyl hydroxyproline, dimethylmethoxy chromanol; bioflavonoid hesperidin olive leaf extract, ubiquinone, super-oxide dismutase, flavanols, isoflavones, furfuryladenine, panthenol, lipoic acid, niacinamide, melatonin, catalase, glutathione, polyphenols, cysteine, allantoin, kinetin, squalane, grape seed extract and camellia sinensis extract, ascorbic acid and its derivatives (ascorbyl palmitate, magnesium ascorbyl phosphate, sodium ascorbyl phosphate) vitamin E and its derivatives (e.g. α-tocopherol, δ-tocopherol, γ-tocopherol, tocopheryl acetate, a hydrophilic vitamin E analog such as 6-Hydroxy-2,5,7,8-tetramethylchroman-2-carboxylic acid (Trolox™), alpha-tocopherol acetate, alpha-tocopheryl polyethylene glycol succinate, alpha-tocopherol palmitate), butylated hydroxytoluene (BHT), butylated hydroxyanisole (BHA), hypophosphorous acid, monothioglycerol, potassium metabisulfite, propyl gallate, sodium bisulfite, sodium formaldehyde sulfoxylate, sodium metabisulfite, sodium sulfite, sodium thiosulfate and sulfur dioxide (see USP-NF and Nema, 1997).

Further non-limiting pharmaceutically suitable materials that may be incorporated in pharmaceutical preparations of the present invention include one or more of enteric coatings, absorption enhancers, pH adjusting agents and buffers, osmolarity adjusters, isotonic agents, preservatives, stabilizers, surfactants, thickening agents, co-solvents, emollients, dispersing agents, coloring agents and wetting agents and ligands/pilote/targeting molecules. Methods for preparing appropriate formulations are well known in the art (see e.g., Hendrickson, 2005).

In cases where parenteral administration is elected as the route of administration, pharmaceutical compositions of the present invention may be provided to patients in combination with additional pharmaceutically acceptable sterile aqueous or non-aqueous solvents, suspensions or emulsions. Examples of non-aqueous solvents are alcohol, benzyl benzoate, canola oil, corn oil, cottonseed oil, N,N-dimethylacetamide, glycerin, mineral oil, peanut oil, olive oil polyethylene glycol, propylene glycol, sesame oil, safflower oil, soybean oil, vegetable oil (see Nema, 1997). Aqueous solvents include water, water-alcohol solutions, emulsions or suspensions, including saline and buffered medical parenteral vehicles including sodium chloride solution, Ringer's dextrose solution, dextrose plus sodium chloride solution, Ringer's solution containing lactose or fixed oils. Intravenous vehicles may include fluid and nutrient replenishers, electrolyte replenishers, such as those based upon Ringer's dextrose, and the like.

In cases where oral administration is elected as the route of administration, pharmaceutical compositions of the present invention may be provided to patients in an encapsulated form such as a soft shell encapsulation. Enteric coatings can further be used on capsules of the present invention to resist prolonged contact with the strongly acidic gastric fluid, but dissolve in the mildly acidic or neutral intestinal environment. Without being so limited, cellulose acetate phthalate, Eudragit™ and hydroxypropyl methylcellulose phthalate (HPMCP) can be used in enteric coatings of pharmaceutical compositions of the present invention. Cellulose acetate phthalate concentrations generally used are 0.5-9.0% of the core weight. The addition of plasticizers improves the water resistance of this coating material, and formulations using such plasticizers are more effective than when cellulose acetate phthalate is used alone. Cellulose acetate phthalate is compatible with many plasticizers, including acetylated monoglyceride, butyl phthalybutyl glycolate, dibutyl tartrate, diethyl phthalate, dimethyl phthalate, ethyl phthalylethyl glycolate, glycerin, propylene glycol, triacetin, triacetin citrate and tripropionin. It is also used in combination with other coating agents such as ethyl cellulose, in drug controlled-release preparations.

Formulations suitable for oral administration can consist of (a) liquid formulations, such as an effective amount of active agent(s)/composition(s) suspended in diluents/solubilizers, such as water, vegetable or animal oils, saline or PEG 400; (b) capsules such as soft shell capsules, sachets or tablets, each containing a predetermined amount of the active ingredient, as liquids, solids, granules or gelatin; (c) suspensions in an appropriate liquid; and (d) suitable emulsions.

In case where the oral formulation of the present invention is a syrup, the syrup is preferably an oil-based syrup and may comprises additional components such as one or more antioxidants. The oil-based syrup can comprise one or more vegetable or animal oils or a combination thereof.

Aqueous solutions suitable for oral use are prepared by dissolving the active compound(s)/composition(s) in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents. Examples of non-aqueous solvents are alcohol, benzyl benzoate, butyl alcohol, polyethylene glycol, propylene glycol, N,N-dimethylacetamide, ethyl oleate, oleyl oleate, glyceryl trioleate, glyceryl dioleate, glyceryl monooleate, cetyl alcohol, stearyl alcohol, capric acid, undecenoic acid, undecanoic acid, lauric acid, oleic acid, synthetic glycerides of saturated fatty acids with 8 to 12 carbon atoms, polyoxyethylene derivatives of glycerol, bees' wax, glycerin, mineral oil, vegetable oil such as but not limited to corn oil, cottonseed oil, peanut oil, canola oil, sesame oil, safflower oil, soybean oilarachis oil, castor oil, linseed oil, soya bean oil, sunflower seed oil, olive oil, fish liver oil, and any combination thereof (see Nema, 1997).

The terms “preservative agent” as used herein are meant to refer to any ingredient capable of retarding or preventing microbial or chemical spoilage and protecting against discoloration. Without being so limited, they include benzalkonium chloride, benzethonium chloride, benzyl alcohol, butylparaben, chlorobutanol, chlorocresol, cresol, ethylparaben, methylparaben, myristyl gamma-picolinium chloride, phenol, phenoxyethanol, phenylmercuric acetate, phenylmercuric nitrate, propylparaben, thimerosal (see Nema, 1997).

The terms “isotonic agent” as used herein are meant to refer to ingredients capable of adjusting osmolarity. Without being so limited, they include dibasic sodium phosphate, sodium bicarbonate, calcium chloride, potassium chloride, sodium lactate, glycerol, sorbitol, xylitol, sodium chloride, dextrose, a Ringer's solution, a lactated Ringer's solution and a mixture of dextrose and a mixture thereof (see relevant sections of USP-NF). A lactated Ringer's solution is a solution of recently boiled distilled water containing 39 mmol/L of sodium ion, 42 mmol/L of chloride ion, 0.6 mmol/L of bicarbonate ion, 1.4 mmol/L of potassium ion and 42 mmol/L of calcium ion—the same concentrations as their occurrence in body fluids. Ingredients are: NaCl 2.25 g, KCl 0.105 g, CaCl₂ 0.12 g, NaHCO₃ 0.05 g.

The term “solvent” as used herein is meant to refer to ingredients capable of facilitating the solubilization of an active sesquiterpene within the formulation. Without being so limited, it includes water, water-alcohol solutions, emulsions or suspensions, including saline and buffered medical parenteral vehicles including sodium chloride solution, Ringer's dextrose solution, dextrose plus sodium chloride solution, Ringer's solution containing lactose, or fixed oils. Intravenous vehicles may include fluid and nutrient replenishers, electrolyte replenishers, such as those based upon Ringer's dextrose, and the like.

In certain embodiments, the present invention encompasses the use of an inert or noble gas for filling the headspace of a container enclosing a formulation of the present invention. Although argon is used as illustrative embodiment below, any inert or noble gas can be used for this purpose such as helium, neon, krypton, xenon and radon.

In a further aspect, the present invention provides a method of preventing or inhibiting tumor growth comprising contacting said cell with a therapeutically effective amount of the above-mentioned compound. The tumors to which the compound of the present invention can be applied include swellings and true tumors including benign and malignant tumors. Specific examples of such tumors are gliomas such as astrocytoma, glioblastoma, medulloblastoma, oligodendroglioma, ependymoma and choroid plexus papilloma; cerebral tumors such as meningioma, pituitary adenoma, neurioma, congenital tumor, metastatic cerebral tumor; squamous cell carcinoma, lymphoma, a variety of adenomas and pharyngeal cancers resulted from these adenomas such as epipharyngeal cancer, mesopharyngeal cancer and hypopharyngeal cancer; laryngeal cancer, thymoma; mesothelioma such as pleural mesothelioma, peritoneal mesothelioma and pericardial mesothelioma; breast cancers such as thoracic duct cancer, lobular carcinoma and papillary cancer; lung cancers such as small cell carcinoma, adenocarcinoma, squamous cell carcinoma, large cell carcinoma and adenosquamous carcinoma; gastric carcinoma; esophageal carcinomas such as cervical esophageal carcinomas, thoracic esophageal carcinomas and abdominal esophageal carcinomas; carcinomas of large intestine such as rectal carcinoma, S-like (sigmoidal) colon carcinoma, ascending colon carcinoma, lateral colon carcinoma, cecum carcinoma and descending colon carcinoma; hepatomas such as hepatocellular carcinoma, intrahepatic hepatic duct carcinoma, hepatocellular blastoma and hepatic duct cystadenocarcinoma; pancreatic carcinoma; pancreatic hormone-dependent tumors such as insulinoma, gastrinoma, VIP-producing adenoma, extrahepatic hepatic duct carcinoma, hepatic capsular carcinoma, pedal carcinoma, renal pelvic and uretal carcinoma; urethral carcinoma; renal cancers such as renal cell carcinoma (Grawitz tumor), Wilms' tumor (nephroblastoma) and renal angiomyolipoma; testicular cancers or germ cell tumors such as seminoma, embryonal carcinoma, vitellicle tumor, choriocarcinoma and teratoma; prostatic cancer, bladder cancer, carcinoma of vulva; hysterocarcinomas such as carcinoma of uterine cervix, uterine corpus cancer and solenoma; hysteromyoma, uterine sarcoma, villous diseases, carcinoma of vagina; ovarian germ cell tumors such as dysgerminoma, vitellicle tumor, premature teratoma, dermoidal cancer and ovarian tumors such as ovarian cancer; melanomas such as nevocyte and melanoma; skin lymphomas such as mycosis fungoides, skin cancers such as endoepidermal cancers resulted from skin cancers, prodrome or the like and spinocellular cancer, soft tissue sarcomas such as fibrous histiocytomatosis, liposarcoma, rhabdomyosarcoma, leiomyosarcoma, synovial sarcoma, sarcoma fibroplasticum (fibrosarcoma), neurioma, hemangiosarcoma, fibrosarcoma, neurofibrosarcoma, perithelioma (hemangiopericytoma) and alveolar soft part sarcoma, lymphomas such as Hodgkin lymphoma and non-Hodgkin lymphoma, myeloma, plasmacytoma, acute myelocytic (myeloid) leukemia and chronic myeloid leukemia, leukemia such as adult T-cell leukemic lymphoma and chronic lymphocytic leukemia, chronic myeloproliferative diseases such as true plethora, essential thrombocythemia and idiopathic myelofibrosis, lymph node enlargement (or swelling), tumor of pleural effusion, ascitic tumor, other various kinds of adenomas, lipoma, fibroma, hemangeoma, myoma, fibromyoma and endothelioma.

Other objects, advantages and features of the present invention will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1 shows the structure of beta-caryophyllene ((1R,4E,9S)-4-11,11-trimethyl-8-methylenebicyclo[7.2.0]undec-4-ene, CAS registry number [87-44-5]). Numbering is in accordance with Collado (1989);

FIG. 2 presents the in vivo effect of oral administration of beta-caryophyllene combined with paclitaxel against B16 melanoma-bearing mice between day 7 and day 17;

FIG. 3 compares the concentration in mM of beta-caryophyllene measured at the bottom of a solution containing various solubilizers. An EtOH solution is used as a control (dotted line);

FIG. 4 presents a beta-caryophyllene oxide/beta-caryophyllene ratio obtained following an autoclave sterilization (121° C., 15 min) in the presence of air, argon or nitrogen as headspace and with or without vitamin E. Solutions not sterilized were used as control;

FIG. 5 presents a beta-caryophyllene oxide/beta-caryophyllene ratio in formulations containing vitamin E following autoclave sterilization and accelerated aging;

FIG. 6 presents a beta-caryophyllene oxide/beta-caryophyllene ratio in formulations containing Trolox™ following autoclave sterilization and accelerated aging;

FIG. 7 presents in vivo effect of paclitaxel used alone as compared to paclitaxel combined with beta-caryophyllene (identified as FPL-99), against B16 melanoma-bearing mice. The tumors were visible and measurable on day 6. Treatments by intravenous injections were performed on days 7, 10, 13 (arrow). *Significantly different from paclitaxel (2 mg/kg)+Cremophor-EL; Statistical analysis by U Wilcoxon-Mann-Whitney test and Student t-test. Values of p<0.05 were considered statistically significant. ⁺Significantly different from control (saline); Statistical analysis by U Wilcoxon-Mann-Whitney test and Student t-test. Values of p<0.05 were considered statistically significant;

FIG. 8 presents the in vivo effect of Taxotere™ against Lewis lung cancer-bearing mice. *Significantly different from control (saline); Student t test, p<0.05; Wilcoxon-Mann-Whitney U test, p<0.05;

FIG. 9 presents the in vivo effect of various dosages of a beta-caryophyllene pharmaceutical composition (FPL) against Lewis lung cancer-bearing mice. *Significantly different from control (saline); Student t test, p<0.05; Wilcoxon-Mann-Whitney U test, p<0.05;

FIG. 10 presents the in vivo potentiating effect of various dosages of a beta-caryophyllene pharmaceutical composition (FPL) on a 5 mg/kg dosage of Taxotere™ against Lewis lung cancer-bearing mice. *Significantly different from control (saline); Student t test, p<0.05; Wilcoxon-Mann-Whitney U test, p<0.05;

FIG. 11 presents the in vivo potentiating effect of various dosages of a beta-caryophyllene pharmaceutical composition (FPL) on a 10 mg/kg dosage of Taxotere™ against Lewis lung cancer-bearing mice. *Significantly different from control (saline); Student t test, p<0.05; Wilcoxon-Mann-Whitney U test, p<0.05;

FIG. 12 presents the in vivo potentiating effect of various dosages of a beta-caryophyllene pharmaceutical composition (FPL) on a 15 mg/kg dosage of Taxotere™ against Lewis lung cancer-bearing mice. *Significantly different from control (saline); Student t test, p<0.05; Wilcoxon-Mann-Whitney U test, p<0.05;

FIG. 13 presents the toxicity of various dosages of Taxotere™ in terms of percentage of loss or gain of weight (day 7) of mice with regard to the initial weight (day 1);

FIG. 14 presents the toxicity of various dosages of Formulation A (FPL) in terms of percentage of loss or gain of weight (day 7) of mice with regard to the initial weight (day 1);

FIG. 15 presents the toxicity of various dosages of Formulation A (FPL) with 5 mg/kg Taxotere™ in terms of percentage of loss or gain of weight (day 7) of mice with regard to the initial weight (day 1);

FIG. 16 presents the toxicity of various dosages of Formulation A (FPL) with 10 mg/kg Taxotere™ in terms of percentage of loss or gain of weight (day 7) of mice with regard to the initial weight (day 1);

FIG. 17 presents the toxicity of various dosages of Formulation A (FPL) with 15 mg/kg Taxotere™ in terms of percentage of loss or gain of weight (day 7) of mice with regard to the initial weight (day 1);

FIG. 18 presents the in vivo effect of various dosages of paclitaxel against Lewis lung cancer-bearing mice. *Significantly different from control (saline); Student t test, p<0.05; Wilcoxon-Mann-Whitney U test, p<0.05;

FIG. 19 presents the in vivo effect of various dosages of Formulation A (FPL) against Lewis lung cancer-bearing mice. *Significantly different from control (saline); Student t test, p<0.05; Wilcoxon-Mann-Whitney U test, p<0.05;

FIG. 20 presents the in vivo potentiating effect of various dosages of Formulation A (FPL) with a 10 mg/kg dosage of paclitaxel against Lewis lung cancer-bearing mice. *Significantly different from control (saline); Student t test, p<0.05; Wilcoxon-Mann-Whitney U test, p<0.05;

FIG. 21 presents the in vivo potentiating effect of various dosages of Formulation A (FPL) on a 20 mg/kg dosage of paclitaxel against Lewis lung cancer-bearing mice. *Significantly different from control (saline); Student t test, p<0.05; Wilcoxon-Mann-Whitney U test, p<0.05;

FIG. 22 presents the in vivo potentiating effect of various dosages of Formulation A (FPL) with a 30 mg/kg dosage of paclitaxel against Lewis lung cancer-bearing mice. *Significantly different from control (saline); Student t test, p<0.05; Wilcoxon-Mann-Whitney U test, p<0.05;

FIG. 23 presents the in vivo potentiating effect of various dosages of Formulation A (FPL) combined with various dosages of paclitaxel against Lewis lung cancer-bearing mice in terms of tumor weight: weight of tumor on day 18 (see Table 12 for description of treatments); ^{†}Significantly different from control (saline); Wilcoxon-Mann-Whitney U test, p<0.05 Significantly different from control (saline); Student t test, p<0.05; and

FIG. 24 presents the toxicity of various dosages of Formulation A (FPL) with various dosages of paclitaxel in terms of mice mean weight on day 0 to day 18; and

FIG. 25 presents the potentiating effect of beta-caryophyllene in ethanol (40-150 mM) on four antitumor agents on a pancreatic tumor cell line Panc 05.04 wherein a combination index (CI) over 1 shows an antagonistic effect, a CI equal to 1 shows an additive effect and a CI lower than 1 shows a potentiating effect.

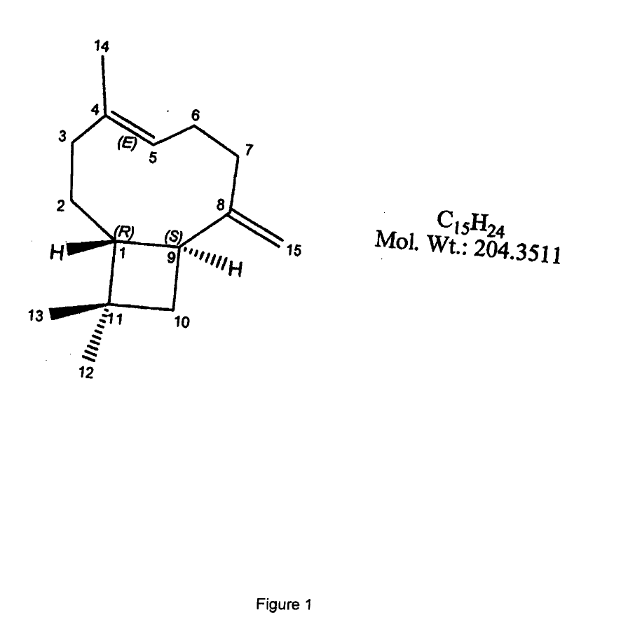

### DESCRIPTION OF ILLUSTRATIVE EMBODIMENTS

Lewis lung carcinoma cells were inoculated subcutaneously in C56BL/6 mice on day 0. The treatments by intravenous injections (tail vein) were performed on days 1 to 4. Mice of each group (n=10) were treated with saline, various dosages of a beta-caryophyllene pharmaceutical composition (FPL), various dosages of Taxotere™ (docetaxel), various dosages of paclitaxel, various dosages of a beta-caryophyllene pharmaceutical composition (FPL) combined with various dosages of Taxotere™ or with various dosages of paclitaxel. Antitumor activity of each treatment was evaluated according to the Calculated Tumor Weight (CTW). The results obtained show that the beta-caryophyllene pharmaceutical composition of the present invention potentiates Taxotere™ and paclitaxel's activities in vivo.

### Example 1

### Oral Administration of Beta-Caryophyllene Formulation

Paclitaxel has a poor bioavailability caused by its high affinity for the mdr1 P-glycoprotein drug efflux pump, which is abundantly present in the gastrointestinal tract (Sparreboom, 1996). Oral administration of paclitaxel alone does not therefore achieve sufficient systemic exposure. It was found that it can be administered orally with cyclosporin A, a known inhibitor of the mdr1 P-glycoprotein, which sufficiently increases its bioavailability (Terwogt, 1999). Beta-caryophyllene also increases paclitaxel's bioavailability by promoting the intracellular accumulation of paclitaxel. The beta-caryophyllene-paclitaxel combination was thus tested orally.

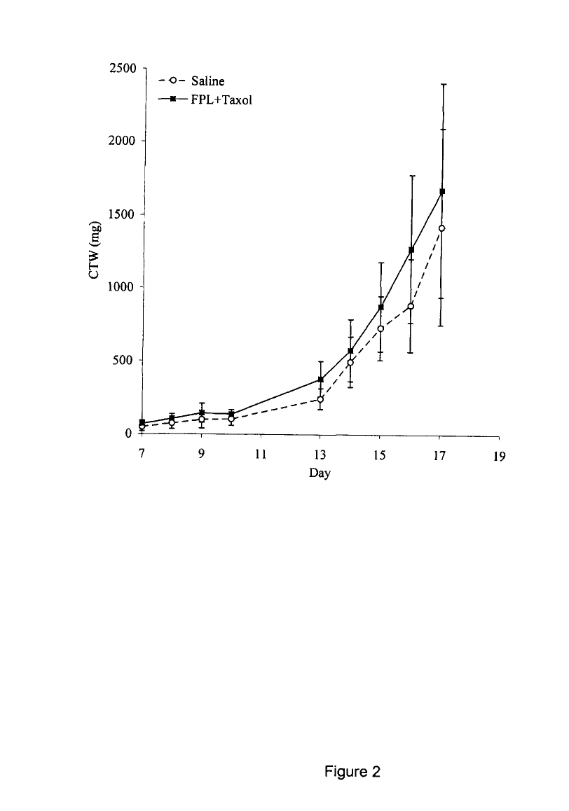

Mice were fed on Day 7, 10 and 13 following injection of B16 tumors with 200 μl of saline (control) or with a volume of 150 to 200 μl of a solution containing: paclitaxel (Taxol™) (160 mg/kg), beta-caryophyllene (50%), ethanol (48%) and polysorbate (2%). As is apparent in FIG. 2, the combination of paclitaxel and beta-caryophyllene in ethanol does not significantly inhibit tumor growth after oral administration. Since it has been shown that paclitaxel can be administered orally when its bioavailability is increased, it was concluded that beta-caryophyllene is degraded in the stomach when formulated in ethanol.

### Example 2

### Beta-Caryophyllene Phosphatidylcholine Emulsion

Fifty microlitres of lecithin and 10 μL of beta-caryophyllene were mixed in a 1.5 mL plastic tube. 940 μL of saline (NaCl 0.9%) were added and the mixture was sonicated for 1 minute. A homogenous yellow and opaque emulsion was obtained. Only a small concentration of beta-caryophyllene was dissolved in the formulation and the stability was insufficient.

### Example 3

### Solubility and Homogeneity of Beta-Caryophyllene in Various Solubilizers

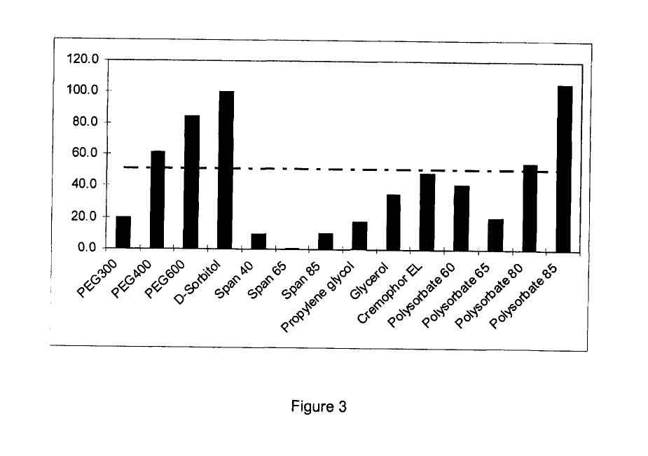

Various solubilizers have been tested in the beta-caryophyllene formulations. Ten mg of beta-caryophyllene and 50 μL or 50 mg of solubilizer were mixed with 950 μL of saline (0.9% NaCl) and sonicated with a 350 watts SONIFIER™ cell disruptor 350 (Sonic Power Co.) for 30 seconds at a power level of 150 W. Visual observation as well as HPLC semi-quantitation were then conducted. Results are presented in Table 1 below and FIG. 3.

TABLE 1
Visual appearance and amount of beta-caryophyllene in each
formulation

| Solubilizing | [Caryo] | |
|---|---|---|
| agent | (mM) | Visual appearance |
| PEG300 | 19.8 | clear transparent |
| PEG400 | 61.2 | clear transparent |
| PEG600 | 84.0 | clear transparent |
| D-Sorbitol | 100.2 | clear transparent |
| Propylene glycol | 18.1 | clear transparent |
| Glycerol | 35.0 | clear transparent |
| Cremophor ™ EL | 48.6 | clear transparent |
| Polysorbate 80 | 55.1 | clear transparent |
| Polysorbate 60 | 41.4 | white translucent |
| Span ™ 40 | 9.6 | white |
| | | heterogeneous |
| Span ™ 65 | 0.9 | white |
| | | heterogeneous |
| Span ™ 85 | 10.4 | white |
| | | heterogeneous |
| Polysorbate 85 | 106.1 | heterogeneous |
| Polysorbate 65 | 20.5 | white opaque |
| EtOH | 51.0 | clear transparent |

Based on the visual observation, polyethylene glycol (PEG), D-sorbitol, propylene glycol, glycerol, Cremophor™ EL and polysorbate 80 produced a clear and transparent solution.

The amount of beta-caryophyllene was then measured in the bottom of each formulation. An amount of beta-caryophyllene in the various solubilizer solutions higher or lower than that measured in the EtOH solution signifies that a gradient is present in the formulation. As shown in FIG. 3, based on this experiment, PEG400, Cremophor™ EL and polysorbate 80 are preferred solubilizers for beta-caryophyllene and other similar sesquiterpenes of interest. Of course, combinations of solubilizers producing beta-caryophyllene gradients when used alone could present homogenous solutions.

### Example 4

### Beta-Caryophyllene, Polysorbate 80 and Sodium Ascorbate

Beta-caryophyllene was surprisingly found to be oxidized in the presence of peroxides found in trace amounts in solubilizers such as polysorbate. Since solubilizers containing polyoxyethylene chains such as polysorbates, PEG, pluronic and Brij™ are susceptible of forming peroxides by radical reactions catalyzed by light and oxygen, it is expected that beta-caryophyllene would also be oxidized in these solubilizers.

Various techniques/compounds were thus tested for their ability to prevent degradation of beta-caryophyllene in the presence of peroxide.

Beta-caryophyllene was combined with polysorbate 80, an aqueous solution of sodium ascorbate and sodium chloride in the proportions described in Table 2 below. The mixture was homogenized with an ultrasound probe (Sonifier cell Disruptor™ 350, Branson Sonic Power Co.) during 5 minutes while maintaining the temperature under 30° C. with an iced water bath. Argon was injected above the solution for 5 minutes so as to remove as much oxygen as possible. After a few days, the clear formulation became yellowish meaning that a degradation had occurred. The mixture was not further analyzed.

TABLE 2

| Compound | Quantity | role |
|---|---|---|
| Beta-Caryophyllene | 100 μL | Active agent |
| Polysorbate 80 | 500 μL | Solubilizer |
| Sodium ascorbate | 100 mg | Antioxidant |
| Saline 0.9% | 9.4 mL | Solvent |

### Example 5

### Sensitivity of Beta-Caryophyllene to Oxidation in Autoclave with and without an Antioxidant

Two batches of drug product formulation were prepared, one containing vitamin E (Formulation A), one without (Formulation B) as described in Table 3 below.

TABLE 3

| Ingredients | Formulation A | | % v/v | Formulation B | % |
|---|---|---|---|---|---|
| β-Caryophyllene | 500 | mg | 1.0 | 500 mg | 1.0 |
| | (0.5 | mL) | | | |
| Polysorbate 80 | 2.5 | mL | 5.0 | 2.5 mL | 5.0 |
| Vitamin E | 50 | mg | 0.1 | — | |
| | (0.05 | mL) | | | |
| Saline 0.9% | 47.5 | mL | 93.9 | 47.5 mL | 94.0 |

The formulations were then flushed in triplicates with either air, nitrogen or argon. They were then sterilized in an autoclave (121° C., 15 min). The sterilization process was monitored with chemical and biological indicators.

After sterilization, the formulation was visually observed. All replicates contained precipitate so that it was not possible to resuspend. However, the following day, the original appearance of formulations was restored by hand shaking.

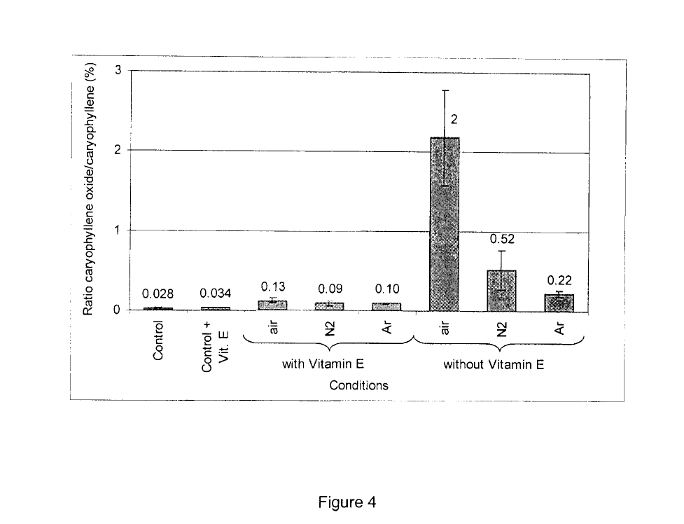

Immediately after the sterilization process, the replicates of each formulation in each condition (air, N₂ or Ar) were analyzed by HPLC-DAD-MS. For each replicate, beta-caryophyllene and beta-caryophyllene oxide concentrations were evaluated and the ratio was calculated. Non sterilized samples of each formulation were also tested on HPLC (controls). Mean values of three replicates are presented in FIG. 4.

It can be noted that oxidation occurred in samples containing vitamin E, but far less than in samples without it. In the vitamin E containing samples, the headspace had no impact. In contrast, without vitamin E, the headspace was found to be significant, argon being the best choice.

### Example 6

### Sensitivity of Beta-Caryophyllene to Oxidation in Autoclave with and without Different Types and Concentrations of Antioxidants

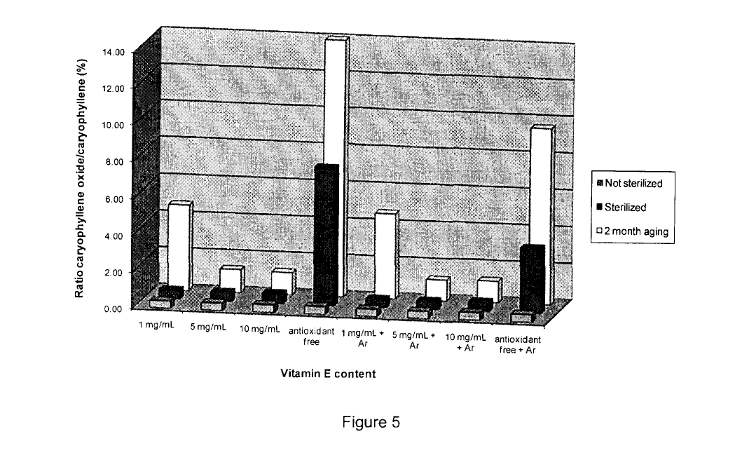

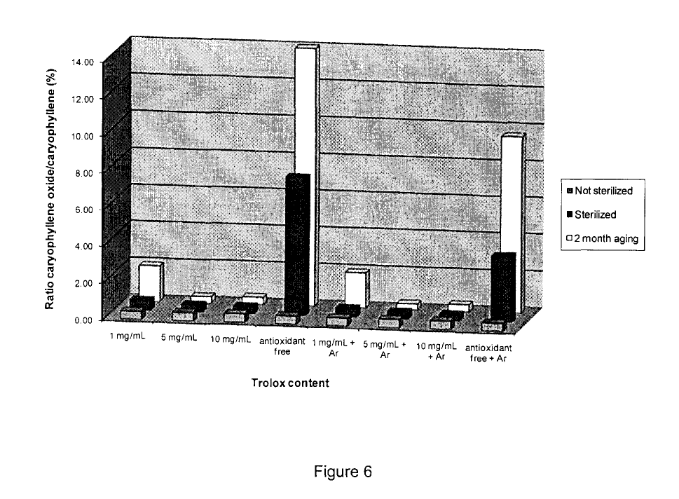

Seven different beta-caryophyllene (10 mg/mL) formulations were prepared: three containing vitamin E (1, 5 and 10 mg/mL), three containing Trolox™ (1, 5 and 10 mg/mL) and one without an antioxidant (the formulations are described in Table 4 below). The headspace of each formulation was then flushed with air or argon. All samples were sterilized with autoclave (121° C., 15 min) and allowed to age at 40° C. For each formulation, beta-caryophyllene and beta-caryophyllene oxide percentages were evaluated by GF-FID and the ratio was calculated. The results obtained for the vitamin E containing formulations and the Trolox™ containing formulations are shown in FIGS. 5 and 6, respectively.

TABLE 4

| | Formulation | | | Formulation | |
|---|---|---|---|---|---|
| | with | | | without | |
| Ingredients | antioxidant | | % v/v | antioxidant | % |
| β-Caryophyllene | 500 | mg | 1.0 | 500 mg | 1.0 |
| Polysorbate 80 | 2.5 | mL | 5.0 | 2.5 mL | 5.0 |
| Vitamin E or | 50 to 500 | mg | 0.1-1.0 | — | — |
| Trolox | | | | | |
| Saline 0.9% | 47.5 | mL | 93.0-93.9 | 47.5 mL | 94.0 |

The beta-caryophyllene (FPL20070131A) used in the formulation contained 0.5% caryophyllene oxide before compounding. This amount was the same in all formulations after compounding as shown by the not sterilized bars in FIGS. 5-6.

Without antioxidant, the beta-caryophyllene could not withstand the sterilization by autoclaving at 121° C. for 15 minutes: up to about 7.5% was degraded. And after 2 months under stress condition (at 40° C.), the degradation of beta-caryophyllene was almost complete (not shown).

When the source of oxygen was removed by replacing the air in the headspace with argon (but without antioxidant in the formulation), the degradation was reduced but still remained important: up to 3.6% after autoclaving and up to 9.6% after 2 months at 40° C.

Although both antioxidants at all tested concentrations provide advantageous results over formulations that do not contain antioxidants, formulations prepared with 5 mg/mL or more of Trolox™ appear to better prevent the oxidation of caryophyllene during the storage process than equivalent amounts of vitamin E. At the same concentration, vitamin E showed higher beta-caryophyllene oxide/beta-caryophyllene ratio than Trolox™. The vitamin E containing formulation is however advantageously clearer than the Trolox™-containing formulation.

These stabilized beta-caryophyllene formulations are emulsions: vitamin E is soluble in the oil phase while Trolox™ is soluble in the aqueous phase. A combination of both a water soluble antioxidant (such as Trolox™, ascorbic acid, hypophosphorous acid, potassium metabisulfite, sodium sulfite) and an oil soluble antioxidant (such as vitamin E), are also beneficial to protect the beta-caryophyllene or other sesquiterpenes from oxidation.

### Example 7

### Preparation of Pharmaceutical Composition Comprising Beta-Caryophyllene

A mixture of vitamin E (100 μL) and polysorbate 80 (500 μL) was first prepared. When the mixture was homogeneous, beta-caryophyllene (100 mg) was added to the mixture, followed by a sodium chloride solution (0.9%, 9.3 mL). The mixture was then mixed using ultrasonic probe at a power level of 150 W with a 350 watts SONIFIER™ cell disruptor 350 (Sonic Power Co.) for 1-2 min, or until a clear and homogeneous solution was obtained.

### Example 8

### In Vivo Potentiating Effect of a Beta-Caryophyllene Formulation on Paclitaxel Against B16-Melanoma Bearing Mice

Formulations: Two formulations were used for this study. The first one was prepared for groups treated with paclitaxel alone while the other one was prepared for the paclitaxel/caryophyllene combination (Table 5). Formulation A was prepared as follows: Different amounts of paclitaxel (0, 2.5, 5 and 10 mg) were dissolved in 500 μL ethanol and 500 μL cremophor-EL. These solutions were further diluted with saline (19 mL) giving final concentrations of paclitaxel of 0, 0.125, 0.25 and 0.5 mg/mL. Formulation B was prepared as follows: beta-caryophyllene (200 μL) was mixed with polysorbate 80 (20 μL), ethanol (780 μL) and different amounts of paclitaxel (0, 2.5, 5 and 10 mg). These solutions were further diluted with 19 mL of a solution containing soya lecithin (2.85 mL), glycerol (0.95 mL) and polysorbate 80 (380 μL) in water (14.82 mL). Each solution was prepared fresh and used within 30 minutes after preparation (see Table 5 below for presenting formulations).

TABLE 5

Formulations

| Ingredients | A | B |
|---|---|---|
| β-caryophyllene | | 1% |
| Paclitaxel | 0, 2.5, 5, 10 mg/mL | 0, 2.5, 5, 10 mg/mL |
| Polysorbate 80 | | 2% |
| Ethanol | 5% | 3.9% |
| Cremophor EL | 5% | |
| Soya lecithin | | 15% |
| Glycerol | | 5% |
| Water | 90% | 73.1% |

Mice: All the experiments were carried out using B6D2F1 male mice, 6-weeks old (Charles Rivers Inc., St-Constant, QC). They were handled and cared for in accordance with the Guide for the Care and Use of Laboratory Animals.

Cells: The B16-F1 mouse skin melanoma cell line was used (#CRL-6323, ATCC). Cells (passages #7 to 21) were grown to 90% confluence in complete MEM medium containing Earle's salts and L-glutamine (Mediatech Cellgro, Va.), 10% fetal bovine serum (Hyclone), vitamins (1×), penicillin (100 I.U./mL) and streptomycin (100 μg/mL), essential amino acids (1×) and sodium pyruvate (1×) (Mediatech Cellgro, Va.). Cells were then harvested using 0.5% trypsin-EDTA (Mediatech Cellgro, Va.). Cells were counted using a hemacytometer and resuspended in MEM medium without SVF. 100 μL of a solution containing 2.5×10⁶ cells/mL per well were inoculated subcutaneously in each mice.

Administration: To establish animal tumor models, 2.5×10⁶ cells were resuspended in 100 μL of MEM medium without SVF (Mediatech Cellgro, Va.) and injected into the subcutaneous tissue of the right flank of the mice on day zero. The tumors were visible and measurable on day 6. Treatments by intravenous injections (tail vein) were performed on days 7, 10, 13. The mice of each group (n=8) were treated with 100 μL of: 1) Saline; 2) Formulation A without paclitaxel; 3) Formulation A with paclitaxel (10 mg/mL); 4) Formulation A with paclitaxel (5 mg/mL); 5) Formulation A with paclitaxel (2.5 mg/mL; 6) Formulation B without paclitaxel; 7) Formulation B with paclitaxel (10 mg/mL); 8) Formulation B with paclitaxel (5 mg/mL); 9) Formulation B with paclitaxel (2.5 mg/mL).

Data analysis: Antitumor activity was evaluated according to two parameters as follows:

(a) Calculated tumor weight (CTW): The CTW of each tumor was estimated from two-dimensional measurements performed once a day with a slide calliper, according to the formula (Bissery et al., 1991): CTW (mg)=(L×W²)/2 with L=length in mm and W=width in mm. CTW values were averaged within each group during and after drug treatment over a period of 17 days post-tumor implant. Differences in CTW between treated and control groups or paclitaxel-Cremophor-EL and paclitaxel-beta-caryophyllene groups were analyzed for significance using the U Wilcoxon-Mann-Whitney test and Student t-test. Values of p<0.05 were considered statistically significant.

(b) T/C value: The T/C was calculated during and after drug treatment as the ratio of the mean CTW of drug-treated mice versus controls (Bissery et al., 1991; Funahashi et al., 2001): T/C=(CTW of the drug-treated group on Day X/CTW of the saline control group on Day X)×100.

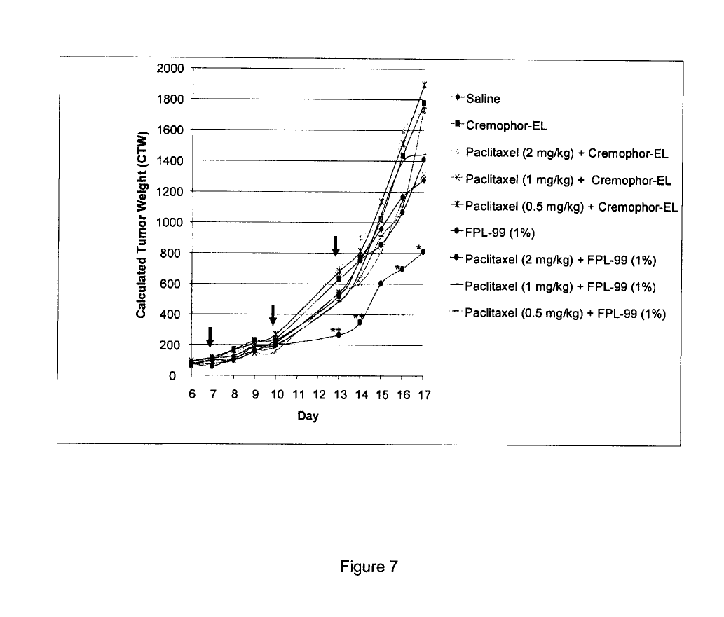

Results presented in FIG. 7 show the time-course of tumor growth using Calculated Tumor Weight (CTW) parameter. After the first treatment on day 7, there is no significant difference between the various groups. However, after the second treatment on day 10, significant differences were observed only between the control (saline) and the groups treated with paclitaxel (2 mg/kg)+beta-caryophyllene (1%). Indeed, this treatment inhibits tumor growth about 54% on day 13 and 51% on day 14. However, no significant difference was observed on day 15, 16 and 17, but CTW was decreasing about 37% on day 15, 40% on day 16 and 37% on day 17. In this experiment, all treatments with paclitaxel I+ cremophor-EL were inactive (no statistically different from control). The statistical difference between the CTW calculated from paclitaxel-beta-caryophyllene and from paclitaxel-cremophor-EL were compared. Statistical analysis shows that CTW from paclitaxel-beta-caryophyllene is significantly lower than paclitaxel-cremophor-EL on day 13 (−63%), 14 (−62%), 16 (−56%) and 17 (−53%).

In Table 6 below, T/C values from beta-caryophyllene, Cremophor-EL, paclitaxel (2 mg/kg)-beta-caryophyllene and paclitaxel (2 mg/kg)-Cremophor-EL were compared on day 13 to 17. A T/C value superior or equal to 100% indicates that the treatment does not inhibit tumor growth. The T/C values of beta-caryophyllene ranged from 89 to 111% and those for Cremophor-EL ranged from 102 to 139%. These results show that Cremophor-EL and beta-caryophyllene do not significantly inhibit tumor growth when used alone. Moreover, T/C values ranging from 106 to 137% were obtained when B16 melanoma-bearing mice were treated with paclitaxel (2 mg/kg)-Cremophor-EL indicating that treatment is inactive. In contrast to paclitaxel (2 mg/kg)-Cremophor-EL, the treatment with beta-caryophyllene combined with paclitaxel inhibit tumor growth with T/C values ranging from 49 to 63%.

TABLE 6
T/C time-course for B16 melanoma tumors treated with
paclitaxel (2 mg/kg) combined or not with beta-caryophyllene (1%).

| | Treated/Control (%) on day | | | | | |
|---|---|---|---|---|---|---|
| Treatment | 13 | 14 | 15 | 16 | 17 | Observations |
| beta- | 95 | 100 | 89 | 91 | 111 | 08/08 animals |
| caryophyllene | | | | | | surviving on day |
| (1%) | | | | | | 17 |
| (n = 8) | | | | | | |
| Cremophor-EL | 116 | 102 | 107 | 123 | 139 | 08/08 animals |
| (n = 8) | | | | | | surviving on day |
| | | | | | | 17 |
| Paclitaxel | 130 | 120 | 106 | 137 | 136 | 08/08 animals |
| (2 mg/kg) + | | | | | | surviving on day |
| Cremophor-EL | | | | | | 17 |
| (n = 8) | | | | | | |
| Paclitaxel | 49 | 46 | 63 | 60 | 63 | 08/08 animals |
| (2 mg/kg) + beta- | | | | | | surviving on day |
| caryophyllene | | | | | | 17 |
| (1%) | | | | | | |
| (n = 8) | | | | | | |

T/C = Treated/Control × 100

Altogether, these results indicate that beta-caryophyllene (1%) and paclitaxel (in cremophor-EL) are not active when used alone against B16 melanoma-bearing mice. However, beta-caryophyllene combined with paclitaxel significantly inhibits tumor growth confirming that beta-caryophyllene potentiates paclitaxel activity in vivo.

### Example 9

### In Vivo Potentiating Effect of Formulation A on Taxotere™ Against Lewis Lung Cancer-Bearing Mice

Cells: The Lewis lung carcinoma cell line was used (#CRL-1642, lot #4372266, ATCC). The cells (passages #9) were grown to 90% confluence in complete DMEM medium containing Earle's salts and L-glutamine (Mediatech Cellgro, Va.), 10% fetal bovine serum (Hyclone), vitamins (1×), penicillin (100 I.U./mL) and streptomycin (100 μg/mL), essential amino acids (1×) and sodium pyruvate (1×) (Mediatech Cellgro, Va.). Cells were then harvested with up and down only. Cells were counted using a hemacytometer and resuspended in DMEM medium without SVF.

Mice: 6-weeks old C57BL/6 male mice were used (Charles Rivers Inc., St-Constant, QC). They were handled and cared for in accordance with the Guide for the Care and Use of Laboratory Animals. To establish animal tumor models, 1×10⁶ cells were resuspended in 100 μL of DMEM medium without SVF and injected into the subcutaneous tissue of the right flank of the mice on day zero.

Inoculation: 100 μL of a solution containing 1×10⁷ cells/mL were inoculated subcutaneously in each mouse.

Treatment Treatments by intravenous injections (tail vein) were performed on days 1 to 4. The mice of each group (n=10) were treated with a volume of 100 μL containing saline, Formulation A of Example 5 above (hereinafter referred to as Formulation A), Taxotere™ or a combination of Formulation A with Taxotere™ in doses presented in Table 7 below:

TABLE 7
Groups of mice treated with saline, Formulation A, Taxotere ™ or a
combination of Formulation A with Taxotere ™

| | | Treatments | | |
|---|---|---|---|---|
| | | | Beta- | |
| | | | caryophyllene | |
| | | | pharmaceutical | |
| | | | formulation | Taxotere ™ |
| Number | Groups | Saline | (mg/kg) | (mg/kg) |
| 1 | A/B | Yes | (—) | (—) |
| 2 | C/D | (—) | (—) | 5 |
| 3 | E/F | (—) | (—) | 10 |
| 4 | G/H | (—) | (—) | 15 |
| 5 | I/J | (—) | 6.25 | (—) |
| 6 | K/L | (—) | 12.5 | (—) |
| 7 | M/N | (—) | 25 | (—) |
| 8 | O/P | (—) | 6.25 | 5 |
| 9 | Q/R | (—) | 6.25 | 10 |
| 10 | S/T | (—) | 6.25 | 15 |
| 11 | U/V | (—) | 12.5 | 5 |
| 12 | W/X | (—) | 12.5 | 10 |
| 13 | Y/Z | (—) | 12.5 | 15 |
| 14 | AA/BB | (—) | 25 | 5 |
| 15 | CC/DD | (—) | 25 | 10 |
| 16 | EE/FF | (—) | 25 | 15 |

Data analysis: Antitumor activity was evaluated according to parameters as follows: (a) Calculated tumor weight (CTW): The CTW of each tumor was estimated from two-dimensional measurements performed once a day with a slide calliper, according to the formula (Bissery et al., 1991): CTW (mg)=(L×W²)/2 with L=length in mm and W=width in mm. CTW values were averaged within each group during and after drug treatment over a period of 17 days post-tumor implant. Differences in CTW between treated and control groups (saline) were analyzed for significance using the U Wilcoxon-Mann-Whitney test and Student t-test. Values of p<0.05 were considered statistically significant. The length and the width of tumor were measured with calliper on day 10 to day 18.

(b) Treated/Control value (T/C) and Tumor Growth Inhibition (TGI): The T/C was calculated during and after drug treatment as the ratio of the mean CTW of TW of drug-treated mice versus controls (Bissery et al., 1991; Funahashi et al., 2001): T/C=(CTW of the drug-treated group on Day X/CTW of the saline control group on Day X)×100. TGI is 100−(T/C) value.

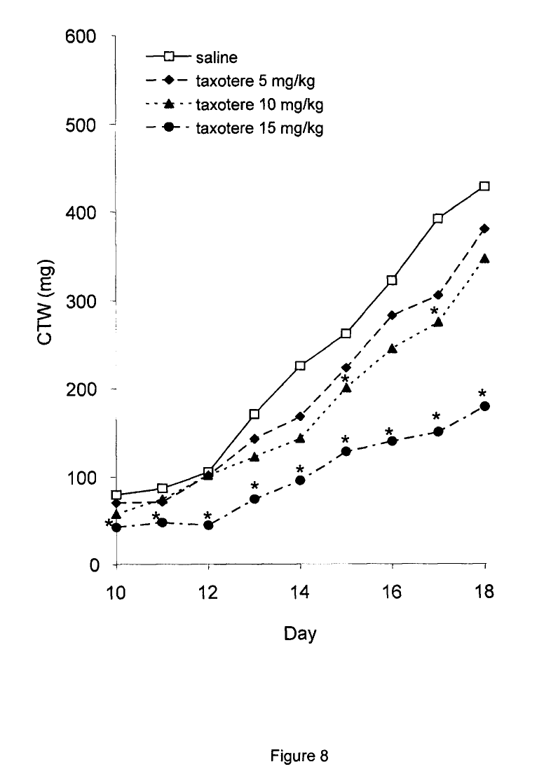

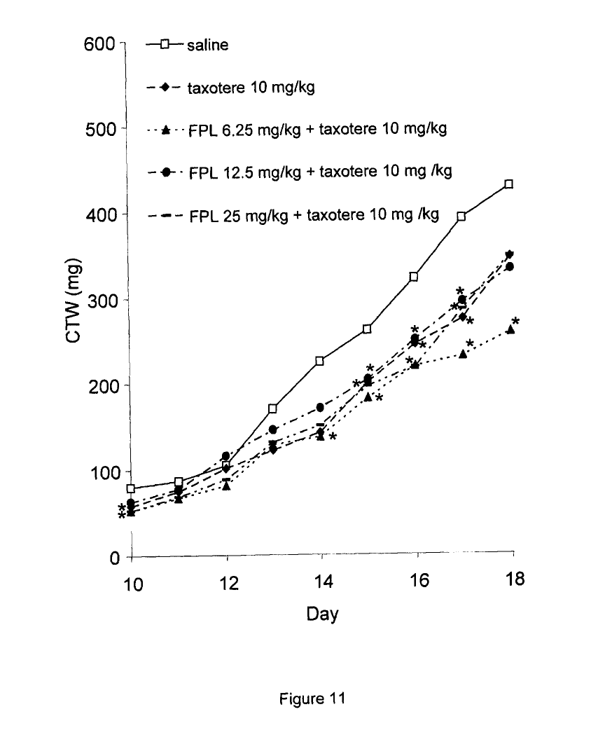

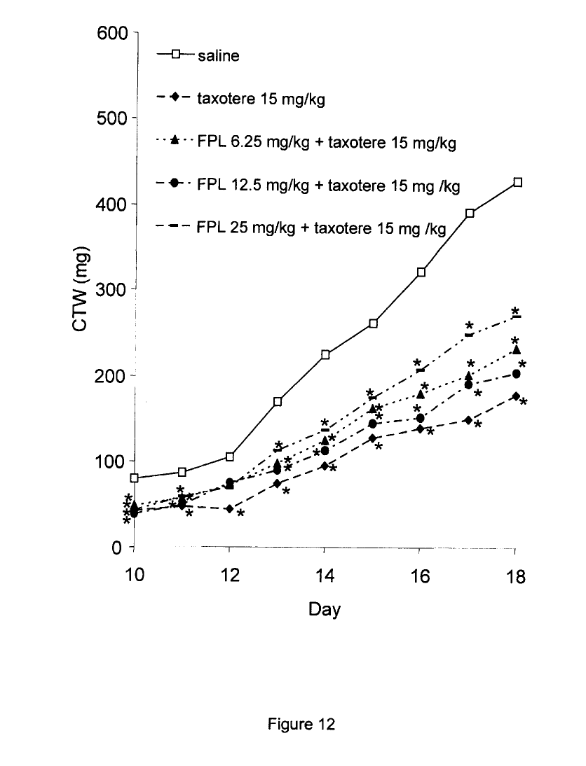

Results are presented in Tables 8 and 9 below and in FIGS. 8-12.

TABLE 8
In vivo potentiating effect of Formulation A combined
with Taxotere ™ against Lewis lung cancer-bearing
mice: tumor volume on day 18 measured with an electronic
calliper; T/C (%) and tumor growth inhibition

| | | CTW | T/C^{a} | TGI^{b} |
|---|---|---|---|---|
| Treatment | Group | (mg) | (%) | (%) |
| Saline | A/B | 428 ± 120 | 100 | (—) |
| Taxotere ™ (5 mg/kg) | C/D | 380 ± 116 | 89 | 11 |
| Taxotere ™ (10 mg/kg) | E/F | 346 ± 107 | 81 | 19 |
| Taxotere ™ (15 mg/kg) | G/H | 178 ± 112^{c} | 42 | 58 |
| Formulation A (6.25 mg/kg) | I/J | 508 ± 126 | 119 | (—) |
| Formulation A (12.5 mg/kg) | K/L | 521 ± 92 | 122 | (—) |
| Formulation A (25 mg/kg) | M/N | 419 ± 135 | 98 | 2 |
| Formulation A (6.25 mg/kg) + | O/P | 281 ± 68^{c} | 66 | 34 |
| Taxotere ™ | | | | |
| (5 mg/kg) | | | | |
| Formulation A (6.25 mg/kg) + | Q/R | 259 ± 66^{c} | 60 | 40 |
| Taxotere ™ | | | | |
| (10 mg/kg) | | | | |
| Formulation A (6.25 mg/kg) + | S/T | 232 ± 54^{c} | 54 | 46 |
| Taxotere ™ (15 mg/kg) | | | | |
| Formulation A (12.5 mg/kg) + | U/V | 472 ± 101 | 110 | (—) |
| Taxotere ™ (5 mg/kg) | | | | |
| Formulation A (12.5 mg/kg) + | W/X | 332 ± 83 | 78 | 22 |
| Taxotere ™ (10 mg/kg) | | | | |
| Formulation A (12.5 mg/kg) + | Y/Z | 204 ± 91^{c} | 48 | 52 |
| Taxotere ™ (15 mg/kg) | | | | |
| Formulation A (25 mg/kg) + | AA/BB | 344 ± 83 | 80 | 20 |
| Taxotere ™ (5 mg/kg) | | | | |
| Formulation A (25 mg/kg) + | CC/DD | 348 ± 91 | 81 | 19 |
| Taxotere ™ (10 mg/kg) | | | | |
| Formulation A (25 mg/kg) + | EE/FF | 271 ± 94^{c} | 63 | 37 |
| Taxotere ™ (15 mg/kg) | | | | |

^{a}T/C: Treated/Control (saline) × 100%
^{b}TGI: Tumor Growth Inhibition = 100 − T/C (%)
^{c}Significantly different from control (saline); Student t test, p < 0.05; Wilcoxon-Mann-Whitney U test, p < 0.05

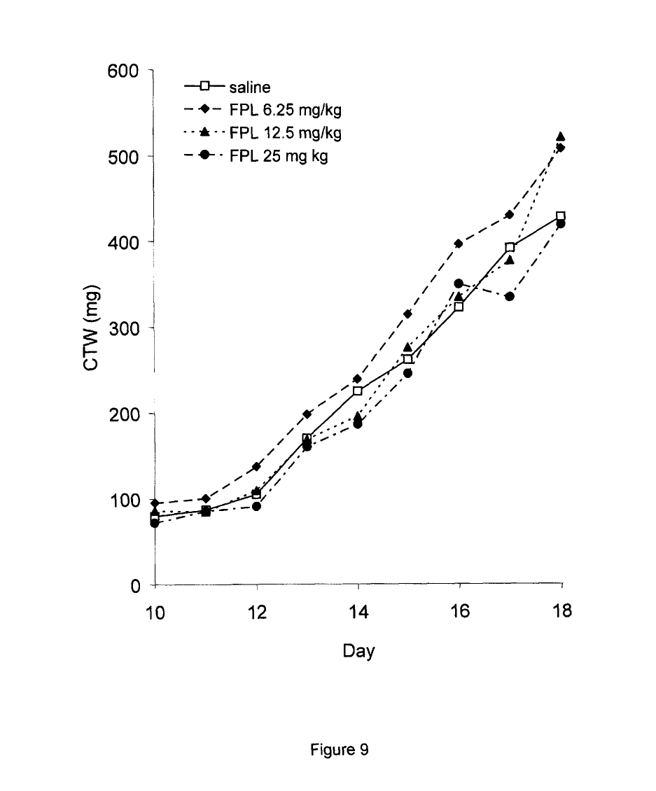

The results presented in FIGS. 8-12 show tumor growth (calculated tumor weight, CTW) from day 10 to day 18. The T/C values and the percentage of tumor growth inhibition (TGI) in comparison with control (saline) were presented in Table 7 above. FIG. 8 shows that Taxotere™ (15 mg/kg) induces a significant inhibition of tumor growth about 58% (on day 18) in comparison with control. The treatment with Taxotere™ 5 mg/kg and 10 mg/kg inhibit tumor growth about 11% and 19% respectively (on day 18), but it is not significantly different from control. FIG. 9 indicates that the three tested Formulation A doses do not significantly affect tumor growth.

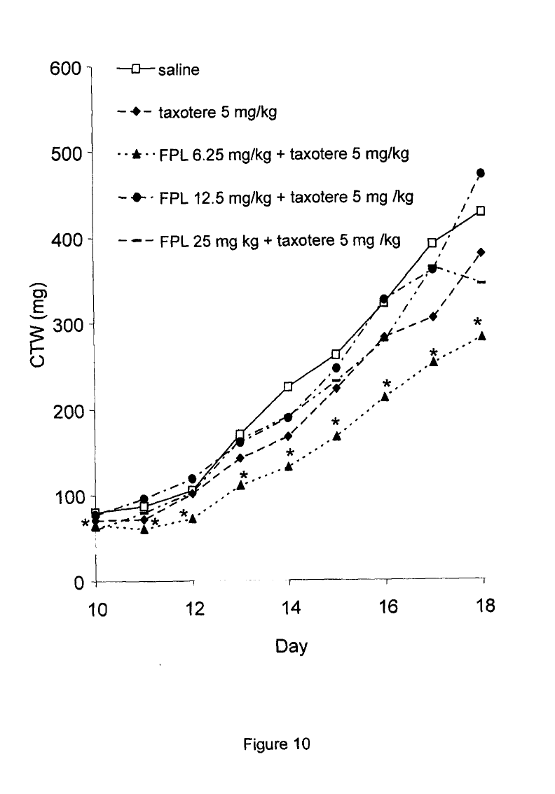

FIGS. 10-12 present growing concentrations of Formulation A injected at the same time as Taxotere™. The results indicate that the best potentiating activity of Formulation A was obtained with the lower dose of Formulation A (6.25 mg/kg). On day 18, Formulation A (6.25 mg/kg) combined with Taxotere™ (10 mg/kg) inhibited significantly tumor growth about 40%. In comparison, Taxotere™ used alone induced tumor growth inhibition about 19%, but the TGI was not significantly different from control. A similar result was obtained when Formulation A (6.25 mg/kg) was combined with 5 mg/kg Taxotere™ (TGI, 36%).

### Example 10

### Toxicity of Formulation A and/or Taxotere™ Treatments on Lewis Lung Cancer-Bearing Mice

The toxicity of the treatments described in Table 7 above was determined using the body weight of mice. The National Cancer Institute considers that a treatment is highly toxic if the loss of weight is superior to 20% with regard to the initial weight. The body weight of the animals was measured every day during 18 days.

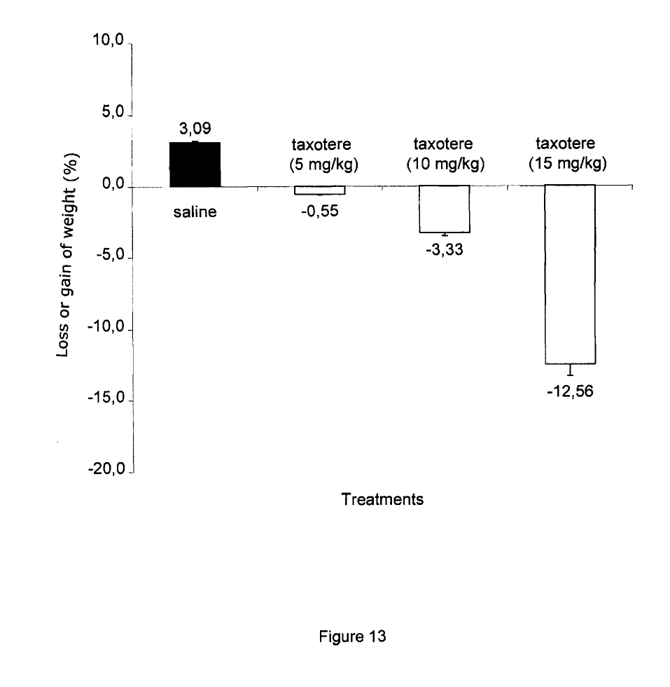

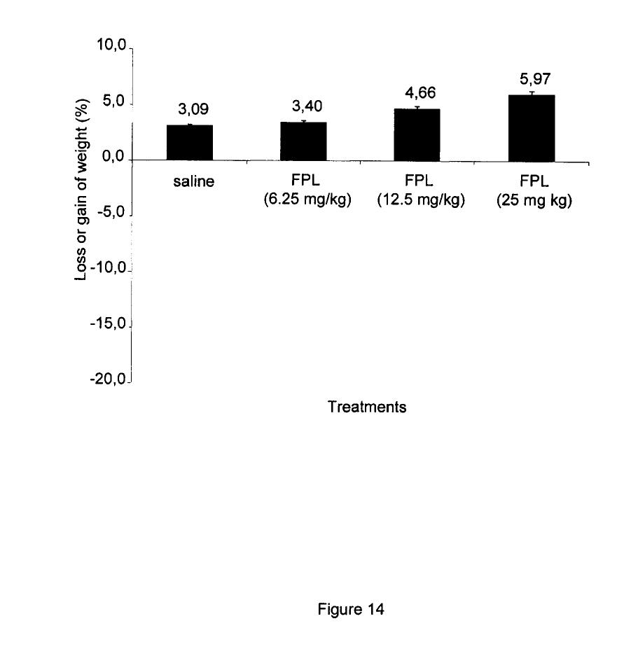

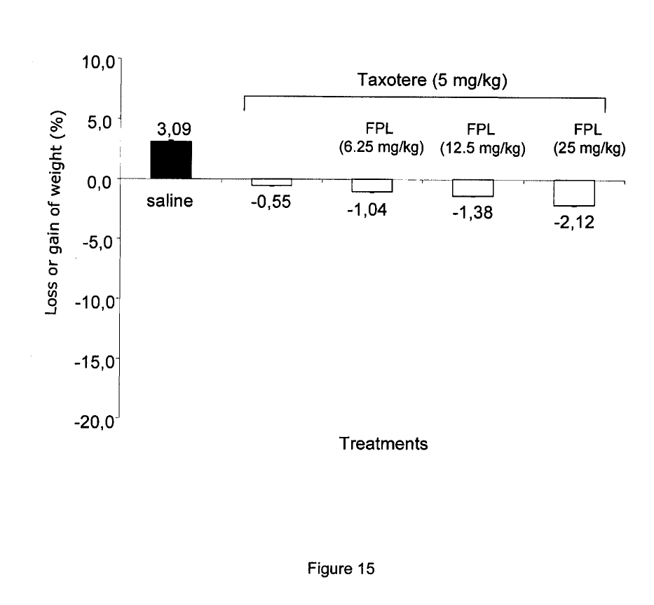

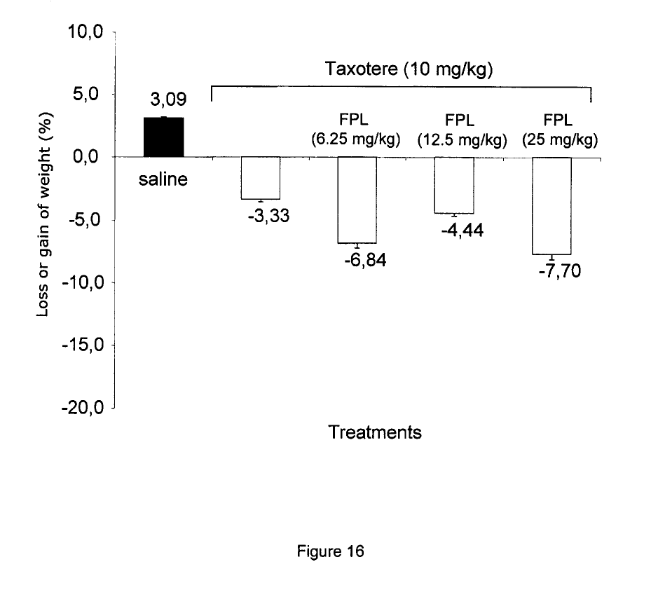

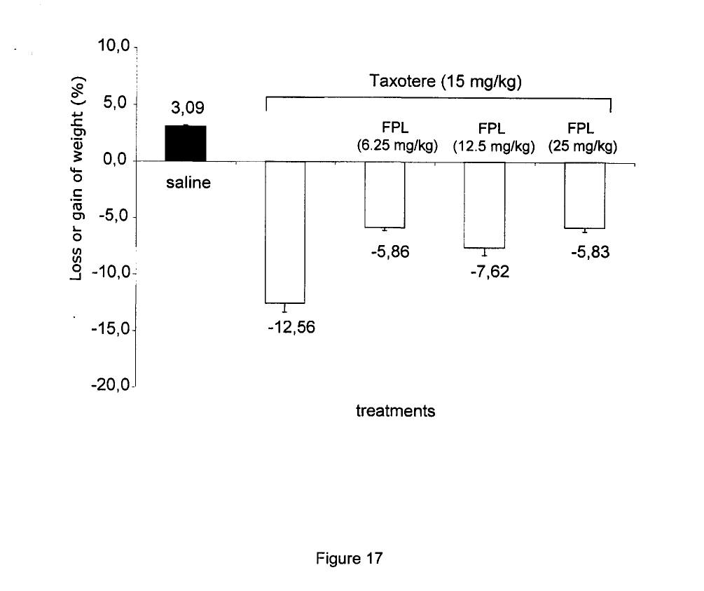

The results are presented in FIGS. 13 to 17 showing the percentage of loss or gain of weight (day 7) with regard to the initial weight (day 1). The treatment with Taxotere™ 5 and 10 mg/kg induced a loss of weight about 0.55% and 3.33% respectively (FIG. 13). In comparison, the control induced a gain of weight of bout 3% with regard to the initial weight. Taxotere™ 15 mg/kg was found to be toxic with loss of weight of about 13% (FIG. 13). Moreover, 4 days following the treatment (day 8), 7 mice on 10 had lost more than 20% of their initial weight (Table 9 below). The treatment with Taxotere™ (15 mg/kg) was significantly effective, however it was highly toxic. Formulation A did not induce loss of weight (FIG. 14). In contrast, earning in weight higher than control was observed in the group of mice tested with Formulation A at dosages of 12.5 mg/kg and 25 mg/kg. In FIGS. 15 and 16, the results showed that Formulation A combined with Taxotere™ 5 and 10 mg/kg slightly increased the loss of weight in comparison with Taxotere™ only. Formulation A combined with Taxotere™ 15 mg/kg significantly decreased the loss of weight in comparison with Taxotere™ only (FIG. 17). Furthermore, Formulation A decreased the toxicity of the Taxotere™ with 3 mice out of 10 (Formulation A 6.25 mg/kg and 12.5 mg/kg) and 1 mice out of 10 (Formulation A; 25 mg/kg) having a loss of weight superior to 20% in comparison with 7 mice on 10 for Taxotere™ only (Table 9 below).

TABLE 9
Toxicity testing using loss of weight of mice on day 1
to day 8

| | | Loss of | |
|---|---|---|---|
| | | weight > 20% | Toxicity* |
| Groups | Treatments | (n = 10) | (%) |
| A/B | saline | 0 | 0 |
| C/D | Taxotere ™ 5 mg/kg | 0 | 0 |
| E/F | Taxotere ™ 10 mg/kg | 0 | 0 |
| G/H | Taxotere ™ 15 mg/kg | 7 | 70 |
| I/J | Formulation A 6.25 mg/kg | 0 | 0 |
| K/L | Formulation A 12.5 mg/kg | 0 | 0 |
| M/N | Formulation A 25 mg/kg | 0 | 0 |
| O/P | Formulation A 6.25 mg/kg + | 0 | 0 |
| | Taxotere ™ 5 mg/kg | | |
| Q/R | Formulation A 6.25 mg/kg + | 1 | 10 |
| | Taxotere ™ | | |
| | 10 mg/kg | | |
| S/T | Formulation A 6.25 mg/kg + | 3 | 30 |
| | Taxotere ™ | | |
| | 15 mg/kg | | |
| U/V | Formulation A 12.5 mg/kg + | 0 | 0 |
| | Taxotere ™ 5 mg/kg | | |
| W/X | Formulation A 12.5 mg/kg + | 1 | 10 |
| | Taxotere ™ | | |
| | 10 mg/kg | | |
| Y/Z | Formulation A 12.5 mg/kg + | 3 | 30 |
| | Taxotere ™ | | |
| | 15 mg/kg | | |
| AA/BB | Formulation A 25 mg/kg + | 0 | 0 |
| | Taxotere ™ 5 mg/kg | | |
| CC/DD | Formulation A 25 mg/kg + | 2 | 20 |
| | Taxotere ™ | | |
| | 10 mg/kg | | |
| EE/FF | Formulation A 25 mg/kg + | 1 | 10 |
| | Taxotere ™ | | |
| | 15 mg/kg | | |

*The treatment was considered to be toxic when the loss of weight was superior to 20% with regard to the initial weight.

### Example 11

### In Vivo Potentiating Effect of Formulation A on Paclitaxel Against Lewis Lung Cancer-Bearing Mice

Cells: The Lewis lung carcinoma cell line was used (#CRL-1642, lot #4372266, ATCC). The cells (passages #9) were grown to 90% confluence in complete DMEM medium containing Earle's salts and L-glutamine (Mediatech Cellgro, Va.), 10% fetal bovine serum (Hyclone), vitamins (1×), penicillin (100 I.U./mL) and streptomycin (100 μg/mL), essential amino acids (1×) and sodium pyruvate (1×) (Mediatech Cellgro, Va.). Cells were then harvested with up and down only. Cells were counted using a hemacytometer and resuspended in DMEM medium without SVF.

Mice: 6-weeks old C57BL/6 male mice were used Charles Rivers Inc., St-Constant, QC). They were handled and cared for in accordance with the Guide for the Care and Use of Laboratory Animals. To establish animal tumor models, 1×10⁶ cells were resuspended in 100 μL of DMEM medium without SVF and injected into the subcutaneous tissue of the right flank of the mice on day zero. Treatments by intravenous injections (tail vein) were performed on days 1 to 4. The mice of each group (n=10) were treated with a volume of 100 μL containing saline, Formulation A (see Example 5), paclitaxel or combination of the Formulation A with paclitaxel.

Inoculation: 100 μL of a solution containing 1×107 cells/mL were inoculated subcutaneously in each mouse.

Treatment: Lewis lung carcinoma cells were inoculated subcutaneous on C57BL/6 mice on day 0. The potentiating effect of the Formulation A combined with paclitaxel was evaluated against Lewis lung carcinoma-bearing mice (C57BL/6). The mice of each group (n=10) were treated with a volume of 100 μL containing saline, Formulation A, paclitaxel or a combination of Formulation A with paclitaxel in doses presented in Table 10 below: The treatments were administered intravenous on day 1 to 4. The length and the width of tumor were measured with calliper on day 8 to day 18. In the end of the experiment (day 18), the tumors were extracted and weighed.

TABLE 10
Groups of mice treated with saline, Formulation A,
paclitaxel or combination of Formulation A with paclitaxel

| | | Treatments | | |
|---|---|---|---|---|
| | | | Formulation A | Paclitaxel |
| Number | Groups | Saline | (mg/kg) | (mg/kg) |
| 1 | A/B | Yes | (—) | (—) |
| 2 | C/D | (—) | (—) | 10 |
| 3 | E/F | (—) | (—) | 20 |
| 4 | G/H | (—) | (—) | 30 |
| 5 | I/J | (—) | 12.5 | (—) |
| 6 | K/L | (—) | 25 | (—) |
| 7 | M/N | (—) | 50 | (—) |
| 8 | O/P | (—) | 12.5 | 10 |
| 9 | Q/R | (—) | 12.5 | 20 |
| 10 | S/T | (—) | 12.5 | 30 |
| 11 | U/V | (—) | 25 | 10 |
| 12 | W/X | (—) | 25 | 20 |
| 13 | Y/Z | (—) | 25 | 30 |
| 14 | AA/BB | (—) | 50 | 10 |
| 15 | CC/DD | (—) | 50 | 20 |
| 16 | EE/FF | (—) | 50 | 30 |

### Data Analysis:

Antitumor activity was evaluated according to the parameters as follows: (a) Calculated tumor weight (CTW): The CTW of each tumor was estimated from two-dimensional measurements performed once a day with a slide calliper, according to the formula (Bissery et al., 1991): CTW (mg)=(L×W²)/2 with L=length in mm and W=width in mm. CTW values were averaged within each group during and after drug treatment over a period of 17 days post-tumor implant. Differences in CTW between treated and control groups (saline) were analyzed for significance using the U Wilcoxon-Mann-Whitney test and Student t-test. Values of p<0.05 were considered statistically significant.

(b) Tumor weight (TW): The TW of each tumor was measured on day 18 after the sacrifice of each mouse. TW values were averaged within each group. Differences in TW between treated and control groups were analyzed for significance using the U Wilcoxon-Mann-Whitney test and Student t-test. Values of p<0.05 were considered statistically significant.

(c) Treated/Control value (T/C) and Tumor Growth Inhibition (TGI): The T/C was calculated during and after drug treatment as the ratio of the mean CTW of TW of drug-treated mice versus controls (Bissery et al., 1991; Funahashi et al., 2001): T/C=(CTW of the drug-treated group on Day X/CTW of the saline control group on Day X)×100. TGI is 100−(T/C) value.

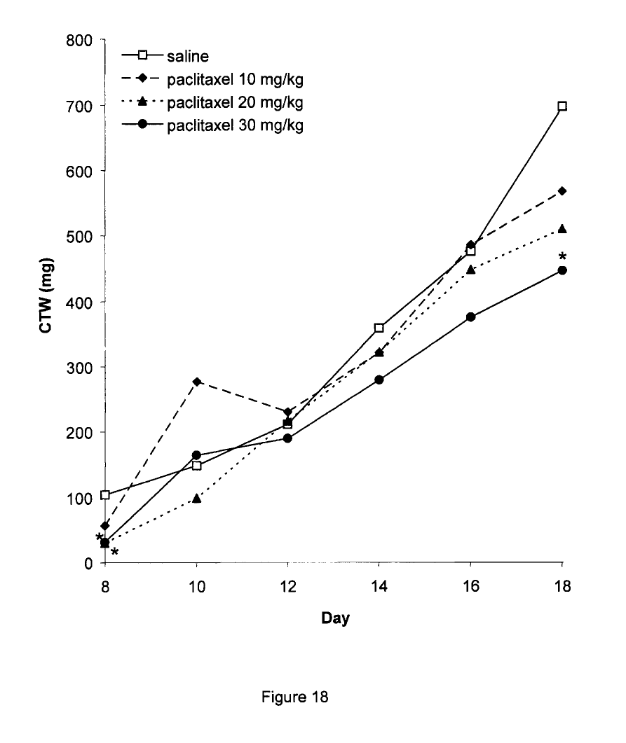

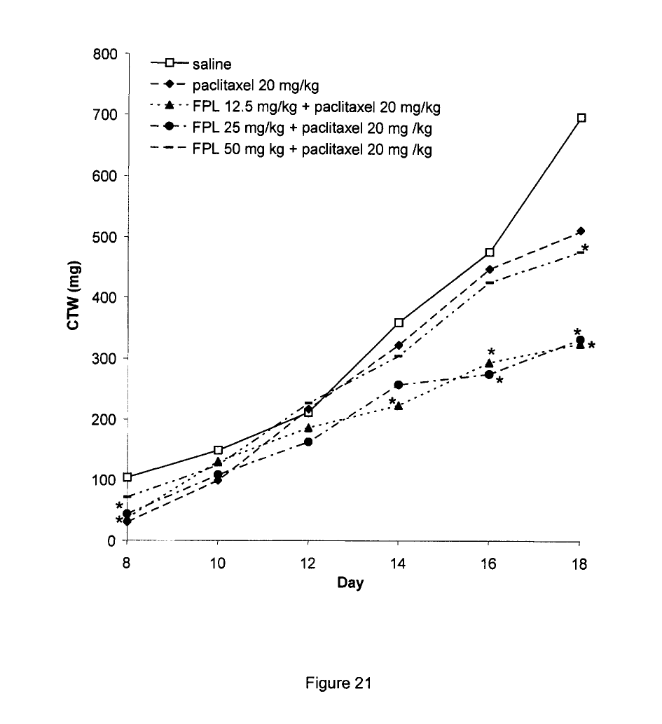

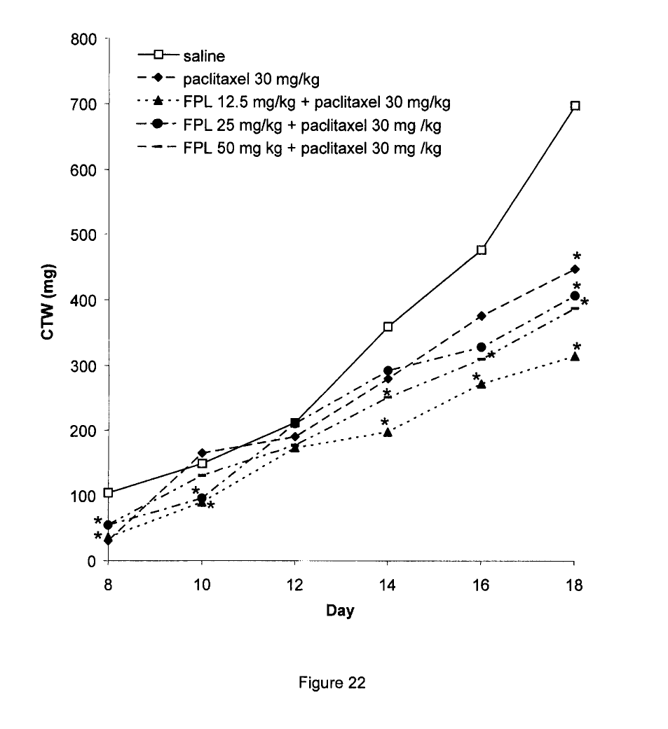

Results are presented in Tables 11 and 12 below and in FIGS. 18-23.

TABLE 11
In vivo potentiating effect of Formulation A combined with paclitaxel
against Lewis lung cancer-bearing mice: calculated tumor weight
(CTW) on day 18 measured with an electronic calliper; T/C (%) and
tumor growth inhibition - Estimated weight

| | | CTW | T/C^{a} | TGI^{b} |
|---|---|---|---|---|
| Treatment | Group | (mg) | (%) | (%) |
| Saline | A/B | 698 ± 125 | 100 | 0 |
| paclitaxel (10 mg/kg) | C/D | 568 ± 155 | 81 | 19 |
| paclitaxel (20 mg/kg) | E/F | 510 ± 274 | 73 | 27 |
| paclitaxel (30 mg/kg) | G/H | 446 ± 118^{c,d} | 64 | 36 |
| Formulation A (12.5 mg/kg) | I/J | 656 ± 252 | 94 | 6 |
| Formulation A (25 mg/kg) | K/L | 638 ± 129 | 92 | 8 |
| Formulation A (50 mg/kg) | M/N | 691 ± 187 | 99 | 1 |
| Formulation A (12.5 mg/kg) + paclitaxel (10 mg/kg) | O/P | 477 ± 118^{c,d} | 68 | 32 |
| Formulation A (12.5 mg/kg) + paclitaxel (20 mg/kg) | Q/R | 324 ± 101^{c,d} | 46 | 54 |
| Formulation A (12.5 mg/kg) + paclitaxel (30 mg/kg) | S/T | 313 ± 133^{c,d} | 45 | 55 |
| Formulation A (25 mg/kg) + paclitaxel (10 mg/kg) | U/V | 520 ± 111^{c,d} | 75 | 25 |
| Formulation A (25 mg/kg) + paclitaxel (20 mg/kg) | W/X | 331 ± 120^{c,d} | 48 | 52 |
| Formulation A (25 mg/kg) + paclitaxel (30 mg/kg) | Y/Z | 406 ± 196^{c,d} | 58 | 42 |
| Formulation A (50 mg/kg) + paclitaxel (10 mg/kg) | AA/BB | 478 ± 188^{c,d} | 68 | 32 |
| Formulation A (50 mg/kg) + paclitaxel (20 mg/kg) | CC/DD | 476 ± 158^{c,d} | 68 | 32 |
| Formulation A (50 mg/kg) + paclitaxel (30 mg/kg) | EE/FF | 387 ± 87^{c,d} | 55 | 45 |

^{a}T/C: Treated/Control (saline) × 100%
^{b}TGI: Tumor Growth Inhibition = 100 − T/C (%)
^{c}Significantly different from control (saline); Wilcoxon-Mann-Whitney U test, p < 0.05
^{d}Significantly different from control (saline); Student t test, p < 0.05

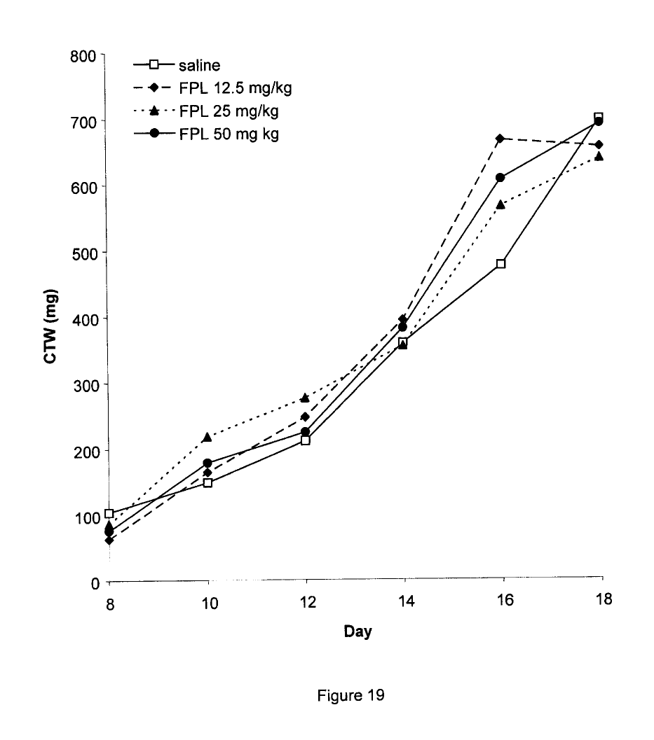

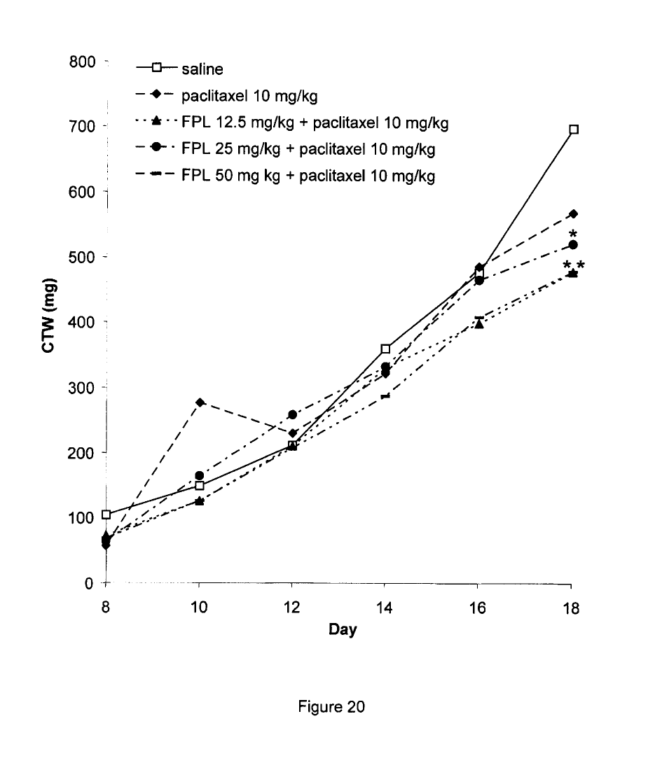

The results presented in FIG. 18-22 show tumor growth (calculated tumor weight, CTW) according to time (day 8 to day 18). The T/C values and the percentage of tumor growth inhibition (TGI) in comparison with control (saline) were presented in Table 11 above. FIG. 18 shows that paclitaxel (30 mg/kg) induces a significant inhibition of tumor growth about 36% on day 18 in comparison with control (saline). In contrast, the treatment with paclitaxel 10 and 20 mg/kg do not inhibit significantly tumor growth. FIG. 19 indicates that three tested Formulation A doses do not significantly affect tumor growth. FIGS. 20-22 present growing concentration of Formulation A combined with 2 dosages of paclitaxel. Altogether, the results indicate that the best potentiating activity of Formulation A was obtained with the lower tested dose of Formulation A (12.5 mg/kg). On day 18, Formulation A (12.5 mg/kg) combined with paclitaxel (20 mg/kg) significantly inhibited tumor growth by about 54% in comparison with no significant TGI (27%) when paclitaxel was used alone. A similar result was obtained with a dose of 25 mg/kg Formulation A (TGI, 52%).

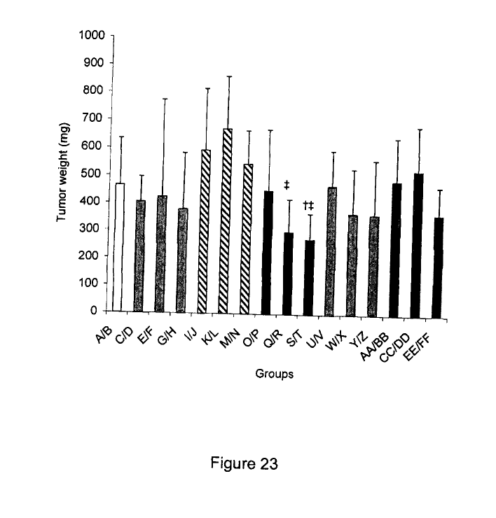

On day 18, the mice were sacrificed and the tumors were extracted and weighed. The real tumor weight results presented in FIG. 23 and Table 12 below confirmed that Formulation A (12.5 mg/kg) (i.e. O/P, Q/R and S/T) is the dosage that best potentiated the antitumor activity of paclitaxel in vivo.

TABLE 12
In vivo potentiating effect of Formulation A combined with paclitaxel
against Lewis lung cancer-bearing mice: tumor weight on day 18;
T/C (%) and tumor growth inhibition - real weight

| | | Tumor weight | T/C^{a} | TGI^{b} |
|---|---|---|---|---|
| Treatment | Group | (mg) | (%) | (%) |
| Saline | A/B | 470 ± 170 | 100 | 0 |
| paclitaxel (10 mg/kg) | C/D | 410 ± 90 | 87 | 13 |
| paclitaxel (20 mg/kg) | E/F | 420 ± 350 | 91 | 9 |
| paclitaxel (30 mg/kg) | G/H | 380 ± 200 | 81 | 19 |
| Formulation A (12.5 mg/kg) | I/J | 590 ± 220 | 127 | (—) |
| Formulation A (25 mg/kg) | K/L | 670 ± 190 | 144 | (—) |
| Formulation A (50 mg/kg) | M/N | 550 ± 120 | 117 | (—) |
| Formulation A (12.5 mg/kg) + paclitaxel (10 mg/kg) | O/P | 450 ± 220 | 96 | 4 |
| Formulation A (12.5 mg/kg) + paclitaxel (20 mg/kg) | Q/R | 300 ± 120^{d} | 64 | 36 |
| Formulation A (12.5 mg/kg) + paclitaxel (30 mg/kg) | S/T | 270 ± 90^{c,d} | 59 | 41 |
| Formulation A (25 mg/kg) + paclitaxel (10 mg/kg) | U/V | 470 ± 130 | 101 | (—) |
| Formulation A (25 mg/kg) + paclitaxel (20 mg/kg) | W/X | 370 ± 160 | 79 | 21 |
| Formulation A (25 mg/kg) + paclitaxel (30 mg/kg) | Y/Z | 370 ± 160 | 79 | 21 |
| Formulation A (50 mg/kg) + paclitaxel (10 mg/kg) | AA/BB | 490 ± 160 | 105 | (—) |
| Formulation A (50 mg/kg) + paclitaxel (20 mg/kg) | CC/DD | 530 ± 160 | 113 | (—) |
| Formulation A (50 mg/kg) + paclitaxel (30 mg/kg) | EE/FF | 370 ± 100 | 79 | 21 |

^{a}T/C: Treated/Control (saline) × 100%
^{b}TGI: Tumor Growth Inhibition = 100 − T/C (%)
^{c}Significantly different from control (saline); Wilcoxon-Mann-Whitney U test, p < 0.05
^{d}Significantly different from control (saline); Student t test, p < 0.05

### Example 12

### Toxicity of Formulation A and/or Paclitaxel Treatments on Lewis Lung Cancer-Bearing Mice

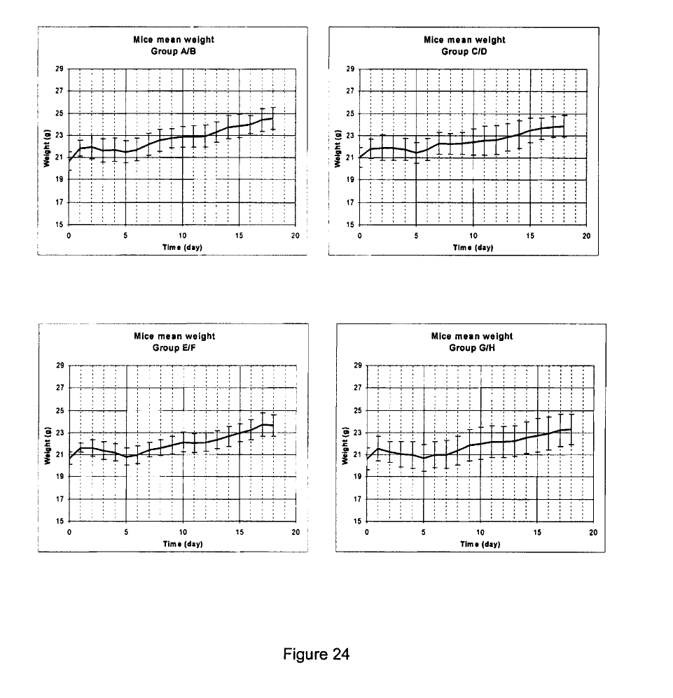

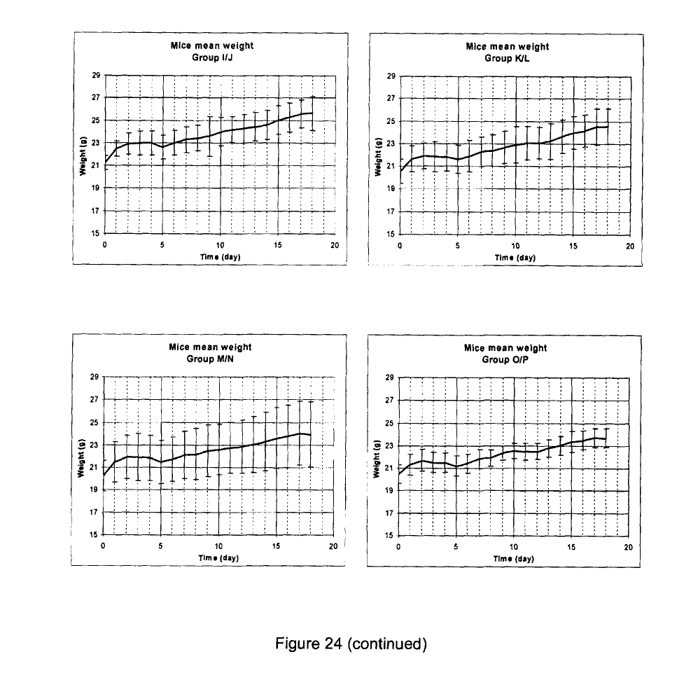

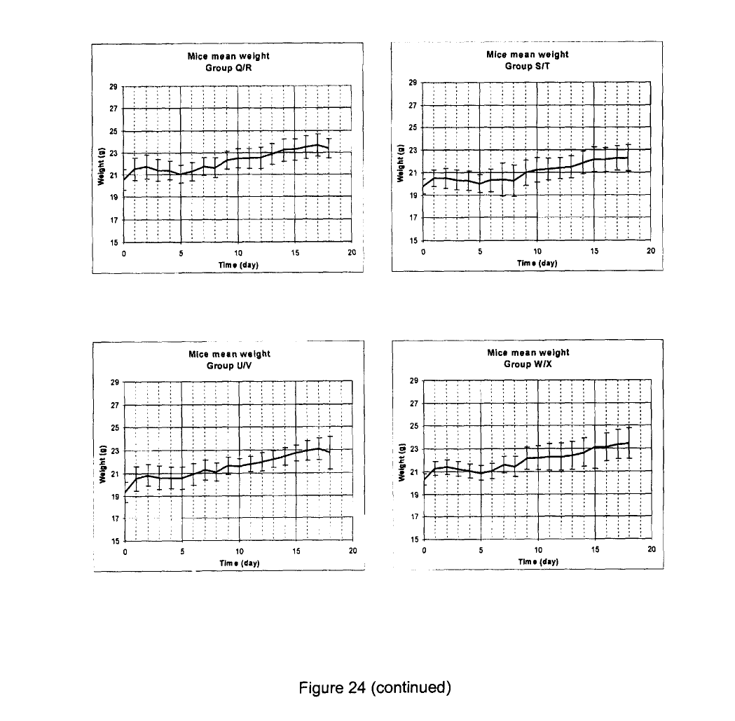

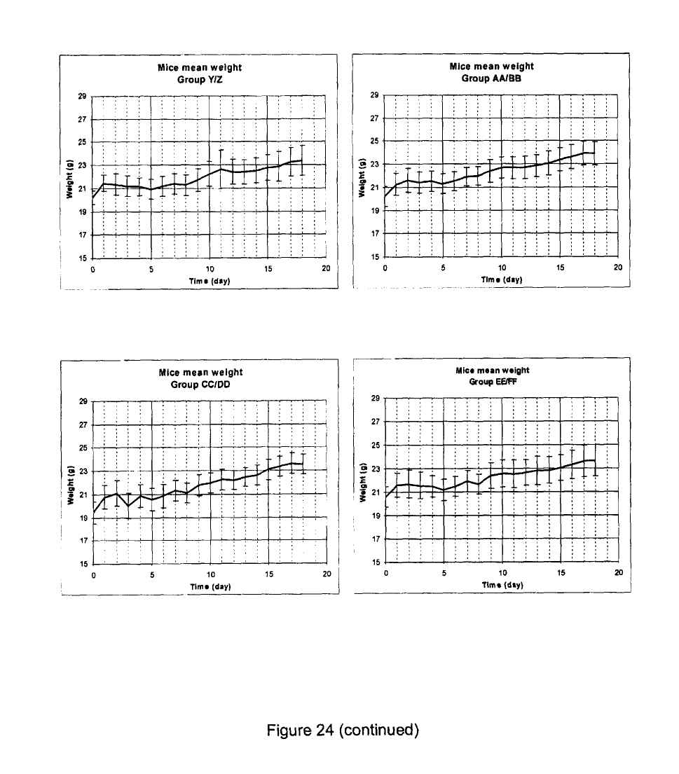

The toxicity of treatments described in Table 10 above was determined by using the weight of mice. The National Cancer Institute considers that a treatment is highly toxic if the loss of weight is superior to 20% of the initial weight. The weights of the animals were measured every day during 18 days. The results are presented in FIG. 24 and indicate that none of the tested treatments led to major toxicity according to this parameter.

### Example 13

### Determination of Maximum Recommended Starting Dose for Human

The maximum recommended starting dose (MRSD) for human was calculated by establishing the No Observed Adverse Effect Level (NOAEL) (see Guidance for Industry and Reviewers. December 2002). Two series of concentrations of Formulation A have been tested on mice, namely formulations comprising 50, 25 and 12.5 mg beta-caryophyllene per kg of mice for potentiating paclitaxel and 25, 12.5, and 6.25 mg beta-caryophyllene per kg of mice for potentiating Taxotere™. The formulation was also tested on rats at 75 mg beta-caryophyllene per kg of rat. No undesirable effects have been observed with any of these doses. The NOAEL is thus 50 mg/kg for mice and 75 mg/kg for rats.

These doses were then scaled up to human equivalent doses (HED) using published conversion tables that take into account the body surface area of each species. Hence the conversion factor from mice to human is 12.3 so that a NOAEL of 50 mg/kg for that species is equivalent to 4.1 mg/kg in human. The conversion factor from rat to human is 6 so that a NOAEL of 75 mg/kg for that species is equivalent to 12.1 mg/kg in human. The largest dose used is that calculated with the most appropriate species. By default, the species in which the lowest HED can be identified is used. The value calculated with the mice dose is thus used.

Then, this value (4.1 mg/kg) was divided by a security factor of 10. The calculated MRSD is thus 0.41 mg/kg. For an average human weighing 60 kg, 24.4 mg is thus injected. When Formulation A of Example 5 is used, 24.4 mg corresponds to 2.44 mL of the formulation.

### Example 14

### Beta-Caryophyllene's Ability of to Potentiate Various Antitumoral Agents on Colon, Lung, Breast, Melanoma, Prostate Overian and Gliobastoma Tumor Cell Lines

Agent: Solutions of each anticancer drug, beta-caryophyllene and tesmilifene were prepared in water, DMSO or ethanol at a concentration of 25 μM to 320 mM, depending on the agent. Tesmilifene is a small molecule that enhances the efficacy (chemopotentiators) of antitumor drugs in breast cancer such as anthracyclines (doxorubicin, epirubicin) and taxanes (Taxotere™ and paclitaxel). The solutions were prepared as follows: beta-caryophyllene (320 mM, ethanol), carboplatin (27 mM, water), carmustine (120 mM, water:ethanol 50:50), chlorambucil (120 mM, ethanol), dacarbazine (80 mM, water), daunorubicin hydrochloride (500 μM, water), doxorubicin hydrochloride (500 μM, water), etoposide (40 mM, DMSO), 5-Fluorouracil (40 mM, water), melphalan (40 mM, ethanol), tamoxifen citrate (80 mM, DMSO), vinblastine sulfate salt (100 μM, DMSO), vincristine sulfate salt (100 μM, water), Taxotere™ anhydrous (25 μM, DMSO), Paclitaxel (50 μM, ethanol), Methotrexate (500 μM, DMSO), Tyrphostin AG 1478 (100 mM, DMSO), Mitoxantrone (2 mM, water) and Cisplatin (34 mM, DMSO). Each solution was prepared fresh and use within 1 hour after preparation. 5 μL of each test article were added to 1 mL of culture medium for a final concentration of 0.5% of solvent and this concentration have no toxic effect on cells.

Cells: The following cell lines were used: DLD-1 (CCL-221, human colon cancer, ATCC); A-549 (CCL-185, human lung cancer, ATCC); MDA-MB-231 (HTB-26, human breast cancer, ATCC); MCF-7 (HTB-22, human breast cancer, ATCC); B16F1 (CRL-6323, murine melanoma, ATCC); SK-MEL-2 (HTB-68, human melanoma, ATCC); PC-3 (CRL-1435, human prostate cancer, ATCC); PA-1 (CRL-1572, human ovary cancer, ATCC); GL-261 (murine glioblastoma); and U-251 (human glioblastoma). Cells were then harvested using trypsine-EDTA. Cells were counted using a hemocytometer and resuspended in DMEM+10% FBS medium. Cells were plated in 96-well microplates (BD Falcon) at a density of 5 to 10×10³ cells per well for Chou and Talalay assay (Chou, 1984) in 100 μL of culture medium and were allowed to adhere for 24 hours before treatment.

Proliferation assay: Increasing concentrations of the anticancer agents and/or beta-caryophyllene were added to 96-well plate (100 μL per well). The final concentration of solvent in the culture medium was maintained below 0.5% (volume/volume) to avoid solvent toxicity. For the plate 1 set, the anticancer drugs were added at a concentration of 2, 1, ½, ¼, ⅛, 1/16, 1/32, 1/64 of IC₅₀ of the anticancer drugs tested (n=6 anticancer drug per plate). For the plate 2 set, the anticancer drugs were added at the same concentrations as with corresponding Plate 1 set and beta-caryophyllene was added to the cells at a concentration of 2, 1, ½, ¼, ⅛, 1/16, 1/32 and 1/64 of its IC₅₀ value. For the plate 3 set, the beta-caryophyllene was added alone at a concentration of 2, 1, ½, ¼, ⅛, 1/16, 1/32, 1/64 of its IC₅₀ value.

The cells were incubated for 48 h at 37° C. and 5% CO₂. Cytotoxicity was assessed using the resazurin reduction test (O'Brien et al., 2000). Fluorescence was measured on an automated 96-well Fluoroskan Ascent FI™ plate reader (Labsystems) using excitation and emission wavelengths of 530 nm and 590 nm, respectively. Resazurin was then removed.

Cytotoxicity was also assessed by DNA quantification using the Hoechst 33342 assay (Richards, 1985) with some modifications. Microplates were frozen at −20° C. overnight. Plates were thawed, a volume of 100 μl of 0.01% SDS in water was added to the wells, the plates were shaken at room temperature for 3 hours and then frozen once more at −20° C. overnight. Plates were thawed again and 100 μl of a solution containing 30 μg/mL Hoechst 33342, 10 mM Tris-HCl, 1 mM EDTA and 4 M NaCl was added to each well. Plates were shaken in the dark (at room temperature) for at least 2 hours, and fluorescence was measured on an automated 96-well Fluoroskan Ascent FI™ plate reader (Labsystems) using excitation and emission wavelengths of 360 nm and 460 nm, respectively.

Data analysis: Cytotoxicity was expressed as the concentration of extract inhibiting cell growth by 50% (GI50). Results were analyzed using the Chou and Talalay method (9) and may be interpreted as follow: Cl>1 represents an antagonist effect of beta-caryophyllene with the corresponding anticancer agent; Cl=1 represents an additive effect of beta-caryophyllene with the corresponding anticancer agent; Cl<1 represents a potentiating effect of beta-caryophyllene with the corresponding anticancer agent.

Results are presented in Tables 13-16 below:

TABLE 13
Testing of 18 anticancer drugs in combination with
beta-caryophyllene on MCF-7 (breast cancer), MDA-MB-231 (breast cancer), A-
549 (lung cancer), DLD-1 (colon cancer) and analyzed by the Chou and Talalay
and method.

| | Mechanism of | Potentiating activity | | | |
|---|---|---|---|---|---|
| Drugs | action | MCF-7 | MDA | A-549 | DLD-1 |
| Carboplatin | Alkylating | (1) | (3) | (1) | (3) |
| (L; G; Bl; O) | agents | | | | |
| Carmustine | | (1) | (3) | (1) | (2) |
| (G; Ly; M) | | | | | |
| Chlorambucil (L) | | (1) | (1) | (3) | (3) |
| Cisplatine | | (2) | (3) | (2) | (1) |
| Dacarbazine | | (1) | (1) | (2) | (3) |
| Melphalan (O; Ly) | | (2) | (3) | (4) | (3) |
| Daunorubicin | Topoisomerase | (2) | (3) | (2) | (3) |
| Doxorubicin (L; B; Bl) | II inhibitors | (1) | (3) | (2) | (4) |
| Etoposide (L; G; O) | | (1) | (1) | (4) | (3) |
| Mitoxantrone (B; P) | | (1) | (3) | (5) | (5) |
| 5-fluorouracil | RNA/DNA | (1) | (3) | (6) | (3) |
| (C; B; Pr) | antimetabolites | | | | |
| Methotrexate | | (2) | (6) | (4) | (4) |
| (C; B; Bl) | | | | | |
| Paclitaxel (L; B; O) | Antimitotic | (1) | (3) | (3) | (2) |
| Taxotere ™ | agents | (1) | (4) | (3) | (3) |
| (L; B; Pr; O) | | | | | |
| Vincristine (L; G) | | (1) | (3) | (2) | (1) |
| Vinblastine | | (1) | (1) | (1) | (1) |
| (L; B; Bl; Pr) | | | | | |
| Tamoxifen (B; O; M) | kinase | (6) | (5) | (3) | (2) |
| Tyrphostin | inhibitors | (1) | (4) | (3) | (1) |
| All drugs | | 17/18 | 4/18 | 8/18 | 7/18 |
| | | (1) or (2) | (1) or (2) | (1) or (2) | (1) or (2) |
| Drugs used in | | 9/10 | 1/10 | 3/6 | 1/2 |
| therapy | | (1) or (2) | (1) or (2) | (1) or (2) | (1) or (2) |

(1) Very High: CI equal or below 1 at IC20 and Cl below 0.6 to IC50
(2) High: CI below 0.5 at IC50
(3) Moderate: 0.5 < CI < 0.8 at IC50
(4) Weak: 0.8 < CI < 1 at IC50
(5) Additive effect: CI = 1
(6) Antagonist effect: CI > 1
Lung Tumor (L);
Colorectal tumor (C);
Breast tumor (B);
Glial tumor (G);
Pancreas tumor (P);
Bladder Tumor (Bl);
Prostate tumor (Pr);
Renal tumor (R);
Testis tumor (T);
Ovary tumor (O);
Head and Neck tumor (HN);
Leukemia (Le);
Lymphoma (Ly);
Melanoma (M)

TABLE 14
Testing of 18 anticancer drugs in combination with
beta-caryophyllene on PC-3 (prostate cancer), PA-1 (ovary cancer), MEL-2
(human melanoma), B16 (murine melanoma) and analyzed by the Chou and
Talalay and method.

| Drugs | | | | | |
|---|---|---|---|---|---|
| (tumors for | | | | | |
| which the | | | | | |
| antitumoral is | | | | | |
| known to be | Mechanism of | Potentiating activity | | | |
| efficient) | action | PC-3 | PA-1 | MEL-2 | B16 |
| Carboplatin | Alkylating agents | (6) | (3) | (3) | (1) |
| (L; G; Bl; O) | | | | | |
| Carmustine | | (1) | (1) | (1) | (1) |
| (G; Ly; M) | | | | | |
| Chlorambucil (L) | | (1) | (3) | (1) | (1) |
| Cisplatine | | (6) | (2) | (1) | (1) |
| Dacarbazine | | (4) | (1) | (1) | (1) |
| Melphalan | | (3) | (1) | (1) | (1) |
| (O; Ly) | | | | | |
| Daunorubicin | Topoisomerase II | (6) | (1) | (2) | (1) |
| Doxorubicin | inhibitors | (4) | (1) | (1) | (1) |
| (L; B; Bl) | | | | | |
| Etoposide | | (6) | (1) | (3) | (3) |
| (L; G; O) | | | | | |
| Mitoxantrone | | (5) | (2) | (3) | (3) |
| (B; P) | | | | | |
| 5-fluorouracil | RNA/DNA | (2) | (2) | (1) | (1) |
| (C; B; Pr) | antimetabolites | | | | |
| Methotrexate | | (6) | (1) | (1) | (6) |
| (C; B; Bl) | | | | | |
| Paclitaxel | Antimitotic agents | (2) | (2) | (3) | (1) |
| (L; B; O) | | | | | |
| Taxotere ™ | | (2) | (1) | (3) | (1) |
| (L; B; Pr; O) | | | | | |
| Vincristine | | (1) | (1) | (1) | (1) |
| (L; G) | | | | | |
| Vinblastine | | (3) | (1) | (1) | (1) |
| (L; B; Bl; Pr) | | | | | |
| Tamoxifen | kinase inhibitors | (2) | (2) | (1) | (3) |
| (B; O; M) | | | | | |
| Tyrphostin | | (2) | (2) | (1) | (1) |
| All drugs | | 8/18 | 16/18 | 13/18 | 14/18 |
| | | (1) or (2) | (1) or (2) | (1) or (2) | (1) or (2) |
| Drugs used in | | 1/4 | 8/9 | 4/4 | 3/4 |
| therapy | | (1) or (2) | (1) or (2) | (1) or (2) | (1) or (2) |

(1) Very High: potentiating or additive activity to IC20 and CI < 0.6 to IC50
(2) High: CI < 0.5 to IC50
(3) Moderate: 0.5 < CI < 0.8 to IC50
(4) Weak: 0.8 < CI < 1 to IC50
(5) Additive effect: CI = 1
(6) Antagonist effect: CI > 1
Lung Tumor (L);
Colorectal tumor (C);
Breast tumor (B);
Glial tumor (G);
Pancreas tumor (P);
Bladder Tumor (Bl);
Prostate tumor (Pr);
Renal tumor (R);
Testis tumor (T);
Ovary tumor (O);
Head and Neck tumor (HN);
Leukemia (Le);
Lymphoma (Ly);
Melanoma (M)

TABLE 15
Testing of 18 anticancer drugs in combination with beta-caryophyllene
on U-251 (human glioblastoma) and GL-261 (murine glioblastoma)
and analyzed by the Chou and Talalay and method.

| | Mechanism of | Potentiating activity | |
|---|---|---|---|
| Drugs | action | U-251 | GL-261 |
| Carboplatin | Alkylating agents | (3) | (2) |
| (L; G; Bl; O) | | | |
| Carmustine | | (1) | (1) |
| (G; Ly; M) | | | |
| Chlorambucil (L) | | (2) | (6) |
| Cisplatine | | (6) | (6) |
| (G; O; M) | | | |
| Dacarbazine | | (1) | (2) |
| (G; M) | | | |
| Melphalan (O; | | (1) | (1) |
| Ly) | | | |
| Daunorubicin | Topoisomerase II | (1) | (2) |
| Doxorubicin | inhibitors | (1) | (3) |
| (L; B; Bl) | | | |
| Etoposide | | (3) | (2) |
| (L; G; O) | | | |
| Mitoxantrone | | (4) | (1) |
| (B; P) | | | |
| 5- | RNA/DNA | (1) | (3) |
| Fluorouracil(C; B; | antimetabolites | | |
| Pr) | | | |
| Methotrexate | | (2) | (6) |
| (C; B; Bl) | | | |
| Paclitaxel | Antimitotic agents | (1) | (2) |
| (L; B; O) | | | |
| Taxotere ™(L; B; | | (1) | (3) |
| Pr; O) | | | |
| Vincristine (L; G) | | (1) | (1) |
| Vinblastine(L; B; | | (1) | (1) |
| Bl; Pr) | | | |
| Tamoxifen | kinase inhibitors | (1) | (2) |
| (B; O; M) | | | |
| Tyrphostin | | (1) | (3) |
| All drugs | | 14/18 | 11/18 |
| | | (1) or (2) | (1) or (2) |

(1) Very High: potentiating or additive activity to IC20 and CI < 0.6 to IC50
(2) High: CI < 0.5 to IC50
(3) Moderate: 0.5 < CI < 0.8 to IC50
(4) Weak: 0.8 < CI < 1 to IC50
(5) Additive effect: CI = 1
(6) Antagonist effect: CI > 1
Lung Tumor (L);
Colorectal tumor (C);
Breast tumor (B);
Glial tumor (G);
Pancreas tumor (P);
Bladder Tumor (Bl);
Prostate tumor (Pr);
Renal tumor (R);
Testis tumor (T);
Ovary tumor (O);
Head and Neck tumor (HN);
Leukemia (Le);
Lymphoma (Ly);
Melanoma (M)

Combinations of beta-caryophyllene and two other antitumor agents, isocaryophyllene and α-humulene, were also tested as described above with some adaptations. 5×10³ cells per well were allowed to adhere for 16 hours before treatment. The final concentration of solvent in the culture medium was maintained at 0.5% (volume/volume) to avoid toxicity. These combinations were tested on MCF-7. Beta-caryophyllene ranging from 2.5 to 40 μg/ml increases significantly anticancer activity of ∝-humulene and isocaryophyllene on MCF-7. The IC50 of ∝-humulene or isocaryophyllene used alone are respectively of 25 μg/ml and 30 μg/ml, respectively, in comparison with 15 μg/ml and 16 μg/ml, respectively, when combined with 10 μg/ml of beta-caryophyllene.

TABLE 16
Comparative potentiating effect of beta-caryophyllene
and Tesmilifene (DPPE) combined with 5 antitumor classes

| | | | | Topoisomerase | | | | | | | |
|---|---|---|---|---|---|---|---|---|---|---|---|
| | | Alkylating | | II | | RNA/DNA | | Antimitotic | | kinase | |
| | | agents | | inhibitors | | antimetabolites | | agents | | inhibitors | |
| | | Total | | Total | | Total | | Total | | Total | |
| | | (60) | % | (40) | % | (20) | % | (40) | % | (20) | % |
| β-caryo | (1) | 30 | 50 | 13 | 33 | 6 | 30 | 24 | 60 | 7 | 35 |
| | (2) | 9 | 15 | 7 | 18 | 4 | 20 | 6 | 15 | 6 | 30 |
| | (3) | 14 | 23 | 11 | 28 | 3 | 15 | 9 | 23 | 4 | 20 |
| | (4) | 2 | 4 | 4 | 10 | 2 | 10 | 1 | 3 | 1 | 5 |
| | (5) | 0 | 0 | 3 | 8 | 0 | 0 | 0 | 0 | 1 | 5 |
| | (6) | 4 | 7 | 2 | 5 | 5 | 25 | 0 | 0 | 1 | 5 |
| DPPE | (1) | 15 | 25 | 7 | 18 | 6 | 30 | 10 | 25 | 6 | 30 |
| | (2) | 12 | 20 | 5 | 13 | 3 | 15 | 8 | 20 | 5 | 25 |
| | (3) | 14 | 23 | 12 | 30 | 3 | 15 | 12 | 30 | 9 | 45 |
| | (4) | 6 | 10 | 4 | 10 | 2 | 10 | 6 | 15 | 0 | 0 |
| | (5) | 3 | 5 | 2 | 5 | 0 | 0 | 0 | 0 | 0 | 0 |
| | (6) | 10 | 17 | 10 | 25 | 6 | 30 | 4 | 10 | 0 | 0 |

(1) Very High: potentiating or additive activity to IC20 and CI < 0.6 to IC50
(2) High: CI < 0.5 to IC50
(3) Moderate: 0.5 < CI < 0.8 to IC50
(4) Weak: 0.8 < CI < 1 to IC50
(5) Additive effect: CI = 1
(6) Antagonist effect: CI > 1

When tested at their IC₅₀ values, 91% of the drugs exhibited a potentiating effect when combined with beta-caryophyllene and 81% when combined with tesmilifene.

### Example 15

### Ability of Beta-Caryophyllene to Potentiate Various Antitumoral Agents on Pancreatic Tumor Cell Lines

Agent: Solutions of each anticancer drug and beta-caryophyllene were prepared in water, DMSO or ethanol at a concentration of 25 μM to 320 mM, depending on the agent. beta-caryophyllene (320 mM, ethanol), Taxotere™ anhydrous (25 μM, DMSO), Paclitaxel (50 μM, ethanol), camptothecin (200 μM, H₂O) and Irinotecan (29.5 mM, H₂O). Each solution was prepared fresh and use within 1 hour after preparation. 5 μL of each test article were added to 1 mL of culture medium for a final concentration of 0.5% of solvent and this concentration have no toxic effect on cells.

Cells: The Panc 05.04 cell line was used. Cells were harvested using trypsine-EDTA. Cells were counted using a hemocytometer and resuspended in DMEM+10% FBS medium. Cells were plated in 96-well microplates (BD Falcon) at a density of 7.5×10³ cells per well for Chou and Talalay assay in 100 μL of culture medium and were allowed to adhere for 24 hours before treatment.

Proliferation assay: Increasing concentrations of the anticancer agents and/or beta-caryophyllene were added to 96-well plate (100 μL per well). The final concentration of solvent in the culture medium was maintained below 0.5% (volume/volume) to avoid solvent toxicity. For the plate 1 set, the anticancer drugs were added at a concentration of 2, 1, ½, ¼, ⅛, 1/16, 1/32, 1/64 of IC₅₀ of the anticancer drugs tested (n=6 anticancer drug per plate). For the plate 2 set, the anticancer drugs were added at the same concentrations as with corresponding Plate 1 set and beta-caryophyllene was added to the cells at a concentration of 2, 1, ½, ¼, ⅛, 1/16, 1/32 and 1/64 of its IC50 value. For the plate 3 set, the beta-caryophyllene was added alone at a concentration of 2, 1, ½, ¼, ⅛, 1/16, 1/32, 1/64 of its IC50 value.

The cells were incubated for 48 h at 37° C. and 5% CO2. Cytotoxicity was assessed using the resazurin reduction test (8). Fluorescence was measured on an automated 96-well Fluoroskan Ascent FI™ plate reader (Labsystems) using excitation and emission wavelengths of 530 nm and 590 nm, respectively. Resazurin was then removed.

Data analysis: Results were analyzed using the Chou and Talalay method (9) and may be interpreted as follow: Cl>1 represents an antagonist effect of beta-caryophyllene with the corresponding anticancer agent; Cl=1 represents an additive effect of beta-caryophyllene with the corresponding anticancer agent; Cl<1 represents a potentiating effect of beta-caryophyllene with the corresponding anticancer agent.

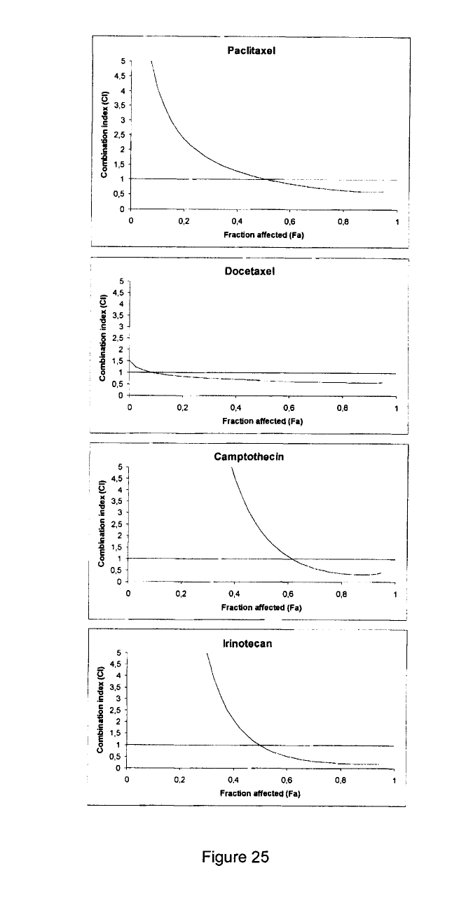

Results with each drug are presented in FIG. 25 and in Table 17 below.

TABLE 17
Testing of 4 anticancer drugs in combination with beta-caryophyllene
on Panc 05.04 (pancreas cancer) and analyzed by the Chou and
Talalay and method.

| | | Potentiating activity |
|---|---|---|
| Drugs | Mechanism of action | Panc 05.04 |
| Irinotecan | Topoisomerase I/II | Potentiating > IC₅₀ |
| Camptothecin | inhibitors | Potentiating > IC₆₀ |
| Paclitaxel | Antimitotic agents | Potentiating > IC₈₀ |
| Docetaxel | | Potentiating > IC₁₀ |

### Example 16

### Testing Formulation A on Animal Models for Other Cancers and for Potentiating Other Antitumoral Agents

Formulation A is tested as described in Examples 9-12 on mice models for the tumors listed in Table 18 below with the antitumoral agents listed in Tables 13-15 above.

TABLE 18

| Cancer origin | | Tumor cell | ATCC |
|---|---|---|---|
| (organs) | Disease | lines | number |
| Prostate | Human | PC-3 | CRL-1435 |
| | adenocarcinoma | | |
| Breast | Human | MCF-7 | HTB-22 |
| | adenocarcinoma | | |
| Breast | Human | MDA-MB-231 | HTB-26 |
| | adenocarcinoma | | |
| Lung (NSCLC) | Human carcinoma | A549 | CCL-185 |
| | (Non-small Cell | | |
| | Lung Cancer) | | |
| Lung (SCLC) | Human carcinoma | DMS 53 | CRL-2062 |
| | (Small Cell Lung | | |
| | Cancer) | | |
| Colon | Human colorectal | DLD-1 | CCL-221 |
| | adenocarcinoma | | |
| Ovary | Human | PA-1 | CRL-1572 |
| | teratocarcinoma | | |
| Brain | Human | U-251 | (—) |
| | glioblastoma | | |
| Brain | Mouse | GI-261 | (—) |
| | glioblastoma | | |
| Skin | Human melanoma | MEL-2 | |
| Skin | Mouse melanoma | B16-F0 | CRL-6322 |
| Pancreas | Human | Panc 05.04 | CRL-2557 |
| | adenocarcinoma | | |
| Liver | Human | Hep G2 | HB-8065 |
| | hepatocellular | | |
| | carcinoma | | |
| Bone marrow | Chronic | K-562 | CCL-243 |
| | myelogenous | | |
| | leukemia (CML) | | |
| Kidney | Human renal cell | 786-O | CRL-1932 |
| | adenocarcinoma | | |
| Stomach | Human gastric | Hs 746T | HTB-135 |
| | carcinoma | | |
| Urinary | Human carcinoma | HT-1376 | CRL-1472 |
| bladder | | | |
| Uterus | Human | KLE | CRL-1622 |
| | adenocarcinoma | | |
| Thyroid | Human carcinoma | SW-579 | HTB-107 |
| B Lymphocyte | Human Burkitt's | DG-75 | CRL-2625 |
| | lymphoma | | |
| Bone | Bone cancer | MG-63 | ORL-1427 |

### Example 17

### Oil-Based Syrup Formulations of Beta-Caryophyllene are Stable and Non-Toxic

Oil-based syrup formulations comprising olive oil as solubilizer, vitamin E (5 mg/ml) and 50 mg/kg to 300 mg/kg of beta-caryophyllene have been tested on mice and shown to be stable and non-toxic.

Although the present invention has been described hereinabove by way of specific embodiments thereof, it can be modified, without departing from the spirit and nature of the subject invention as defined in the appended claims.

### REFERENCES

1. Ha, E.; Wang, W. et Wang, Y. J.; Peroxide formation in polysorbate 80 and protein stability. Journal of Pharmaceutical Sciences, 91(10), 2252-2264 (2002).
2. Guidance for Industry and Reviewers. Estimating the Safe Starting Dose in Clinical Trials for Therapeutics in Adult Healthy Volunteers, U.S. Department of Health and Human Services, Food and Drug Administration, Center for Drug Evaluation and Research (CDER), Center for Biologics Evaluation and Research (CBER), December 2002, Pharmacology and Toxicology.
3. Yamada H, Uchida N, Maekawa R and Yoshioka T, Sequence-dependendent antitumor efficacy of combination chemotherapy with nedapltin, a newly developed platinium, and paclitaxel, Cancer Letters, 172, 17-25, 2001
4. Bissery M C, Guenard D, Gueritte-Voegelein F, Lavelle F (1991). Experimental antitumor activity of Taxotere (RP 56976, NSC 628503), a taxol analogue. Cancer Res 51: 4845-4852.
5. Funahashi Y, Koyanagi N, Kitoh K (2001). Effect of E7010 on liver metastasis and life span of syngeneic C57Bl/6 mice bearing orthotopically transplanted murine colon 38 tumour. CancerChemother Pharmacol 47: 179-184.
6. Nema, S. et al. Excipients and their use in injectable products, PDA J. of Pharm. Science and Technol., 51(4), 166-171 (1997).
7. Chou T-C., Talalay P., 1984, Quantitive analysis of dose-effect relationships: the combined effects of multiple drugs or enzyme inhibitors, Adv. Enxzyme Regul, 22, 27-55.
8. O'Brien J, Wilson I, Orton T, Pognan F., 2000, Investigation of the alamar blue (resazurin) fluorescent dye for the assessment of mammalian cell cytotoxicity. European Journal of Biochemistry 267: 5421-6.
9. Richards W L, Song M K, Krutzsch H, Evarts R P, Marsden E, Thorgeirsson S S., 1985, Measurement of cell proliferation in microculture using Hoechst 33342 for the rapid semiautomated microfluorimetric determination of chromatin DNA. Exp Cell Res, 159:235-46.
10. Terwogt J M M, Malingre M M, Beijnen J H, Huinink W W, Rosing H, Koopman F J, van Tellingen O, Swart M, and Schellens J H M, 1999, Coadministration of Oral Cyclosporin A Enables Oral Therapy with Paclitaxel Clinical Cancer Research 5: 3379-3384.
11. Sparreboom A, Van Asperen J, Mayer U, Schinkel A H, Smit J W, Meijer D K F, Borst P, Nooijen W J, Beijnen J H, Van Tellingen O, 1997 Limited oral bioavailability and active epithelial excretion of paclitaxel (Taxol) caused by P-glycoprotein in the intestine. Proc. Natl. Acad. Sci. USA 94: 2031-2035.
12. Hendrickson, R. Ed. Remington: The Science and Practice of Pharmacy, 21st ed.; Lippincott Williams & Wilkins: Baltimore Md., 2005.

1. A pharmaceutical composition comprising a water insoluble sesquiterpene, one or more antioxidants and one or more solubilizers selected from the group consisting of an oil, PEG400, a derivative of castor oil and ethylene oxide, and polysorbate.

2. The pharmaceutical composition of claim 1, wherein the sesquiterpene is beta-caryophyllene.

3-9. (canceled)

10. The pharmaceutical composition of claim 1, wherein the one or more antioxidants are selected from the group consisting of vitamin E, a hydrophilic vitamin E analog, alpha tocopherol acetate, butylated hydroxytoluene (BHT) and butylated hydroxyanisole (BHA).

11. The pharmaceutical composition of claim 1, wherein the antioxidant is 6-Hydroxy-2,5,7,8-tetramethylchroman-2-carboxylic acid.

12. The pharmaceutical composition of claim 1, wherein the antioxidant is vitamin E.

13. (canceled)

14. The pharmaceutical composition of claim 11, wherein the solubilizer is polysorbate 80.

15. The pharmaceutical composition of claim 11, wherein the solubilizer is a derivative of castor oil and ethylene oxide.

16. (canceled)

17. The pharmaceutical composition of claim 14, further comprising an isotonic agent selected from the group consisting of dibasic sodium phosphate, sodium bicarbonate, calcium chloride, potassium chloride, sodium lactate, glycerol, sorbitol, xylitol, sodium chloride, dextrose, a Ringer's solution, a lactated Ringer's solution and a mixture of dextrose and a mixture thereof.

18. The pharmaceutical composition of claim 2, comprising from about 0.01 mg/mL to about 100 mg/mL of beta-caryophyllene, from about 0.0001% to about 5% v/v of antioxidant, from about 0.01% to about 20% v/v of solubilizer, and an isotonic agent.

19. The pharmaceutical composition of claim 2, comprising about 1% v/v of beta-caryophyllene, about 0.1% v/v of antioxidant, about 5% v/v of solubilizer, and about 93.5% v/v of an isotonic agent.

20. The pharmaceutical composition of claim 19, wherein the antioxidant is vitamin E and the solubilizer is polysorbate 80.

21. The pharmaceutical composition of claim 19, wherein the antioxidant is 6-Hydroxy-2,5,7,8-tetramethylchroman-2-carboxylic acid and the solubilizer is polysorbate 80.

22. The pharmaceutical composition of claim 19, wherein the antioxidant is a combination of 6-Hydroxy-2,5,7,8-tetramethylchroman-2-carboxylic acid and vitamin E.

23. (canceled)

24. The pharmaceutical composition of claim 19, wherein the isotonic agent is sodium chloride.

25-29. (canceled)

30. The pharmaceutical composition of claim 22, wherein said composition is an oil-based syrup.

31-39. (canceled)

40. The pharmaceutical composition of claim 11, further comprising an antitumoral agent.

41-44. (canceled)

45. The pharmaceutical composition of claim 40, wherein the antitumoral agent is an antimitotic selected from the group consisting of paclitaxel and docetaxel.

46-52. (canceled)

53. A method of using the pharmaceutical composition of claim 2 comprising administering the composition to a subject in need thereof prior, simultaneously or after to administration of an antitumoral agent.

54-70. (canceled)

71. A kit comprising the pharmaceutical composition of claim 1 and instructions to use it in combination with an antitumoral agent.

72-77. (canceled)
